SEQUENCE LISTING (82 sequences)

SEQ ID NO: 1 - protein, 7 aa, Artificial Sequence
  Description of Artificial Sequence Synthetic peptide
  MOD_RES (4)..(6): Any amino acid
KENXXXN

SEQ ID NO: 2 - DNA, 21 bp, Homo sapiens
gagagagttctcacatttaat

SEQ ID NO: 3 - DNA, 21 bp, Homo sapiens
actcactagaatattccttat

SEQ ID NO: 4 - DNA, 23 bp, Homo sapiens
gaacgtttagaaatgatttcaaa

SEQ ID NO: 5 - DNA, 21 bp, Homo sapiens
cgtgaacataactgagggcat

SEQ ID NO: 6 - DNA, 21 bp, Homo sapiens
cgcaacaaatagtcggaacat

SEQ ID NO: 7 - DNA, 21 bp, Homo sapiens
cgtgatttgcttagactgttt

SEQ ID NO: 8 - DNA, 21 bp, Homo sapiens
cttagactgtttccacttata

SEQ ID NO: 9 - DNA, 21 bp, Homo sapiens
ctccaaagaaaccatcaagaa

SEQ ID NO: 10 - DNA, 21 bp, Homo sapiens
tggtggtaatgataacttggt

SEQ ID NO: 11 - DNA, 21 bp, Homo sapiens
agaccaacccatcacctcagt

SEQ ID NO: 12 - DNA, 21 bp, Homo sapiens
atgcgcctgaaatccgaaatg

SEQ ID NO: 13 - DNA, 21 bp, Homo sapiens
gcagaaacggcttcgaaatat

SEQ ID NO: 14 - DNA, 21 bp, Homo sapiens
ctaagctggaacagctatatc

SEQ ID NO: 15 - DNA, 21 bp, Homo sapiens
ccagcagagtacccattcaaa

SEQ ID NO: 16 - DNA, 21 bp, Homo sapiens
ccaccgaagatcacatttaaa

SEQ ID NO: 17 - DNA, 21 bp, Homo sapiens
aggtctgtctgccagtaatta

SEQ ID NO: 18 - DNA, 21 bp, Homo sapiens
gaatactctaaggaccgtaaa

SEQ ID NO: 19 - DNA, 21 bp, Homo sapiens
cactttctggcaccgagttta

SEQ ID NO: 20 - DNA, 21 bp, Homo sapiens
tggaacagtatatgaagacct

SEQ ID NO: 21 - DNA, 21 bp, Homo sapiens
cccttacaatgcgcccacagt

SEQ ID NO: 22 - DNA, 21 bp, Homo sapiens
tgtatgatgtcaggaccattc

SEQ ID NO: 23 - DNA, 21 bp, Homo sapiens
cctgcaagaaacctactcaaa

SEQ ID NO: 24 - DNA, 21 bp, Homo sapiens
gcctgtccttgtgtcgtcttt

SEQ ID NO: 25 - DNA, 21 bp, Homo sapiens
caacgatgagaactgtggcat

SEQ ID NO: 26 - DNA, 21 bp, Homo sapiens
gcaggatggcatttaacggat

SEQ ID NO: 27 - DNA, 21 bp, Homo sapiens
ccacatgcattgcatcctcaa

SEQ ID NO: 28 - DNA, 21 bp, Homo sapiens
ccgccaggaatggaagttcaa

SEQ ID NO: 29 - DNA, 21 bp, Homo sapiens
gctgcaacaaggtggaaacaa

SEQ ID NO: 30 - DNA, 21 bp, Mus musculus
cctctcaaagtcctcaataaa

SEQ ID NO: 31 - DNA, 21 bp, Mus musculus
cctcaactatttccgcaatta

SEQ ID NO: 32 - DNA, 62 bp, Homo sapiens
cgtatgtgccttgaactgaattagtgaagccacagatgtaattcagttcaaggcacatacgt

SEQ ID NO: 33 - DNA, 62 bp, Homo sapiens
gggagccatcgcctatgaaattagtgaagccacagatgtaatttcataggcgatggctccca

SEQ ID NO: 34 - DNA, 62 bp, Homo sapiens
gccgcaaatgtgcttcatctatagtgaagccacagatgtatagatgaagcacatttgcggct

SEQ ID NO: 35 - RNA, 19 nt, Artificial Sequence
  Description of Artificial Sequence Synthetic oligonucleotide
gcuuagacuguuuccacuu

SEQ ID NO: 36 - RNA, 21 nt, Artificial Sequence
  Description of Artificial Sequence Synthetic oligonucleotide
cgugaacauaacugagggcau

SEQ ID NO: 37 - RNA, 21 nt, Artificial Sequence
  Description of Artificial Sequence Synthetic oligonucleotide
aaaugugggaugaaaaacuuc

SEQ ID NO: 38 - RNA, 21 nt, Artificial Sequence
  Description of Artificial Sequence Synthetic oligonucleotide
aggucugucugccaguaauua

SEQ ID NO: 39 - RNA, 19 nt, Artificial Sequence
  Description of Artificial Sequence Synthetic oligonucleotide
uucaauaaauucuugaggu

SEQ ID NO: 40 - DNA, 20 bp, Artificial Sequence
  Description of Artificial Sequence Synthetic primer
ccagtgaccatgatagtgtt

SEQ ID NO: 41 - DNA, 21 bp, Artificial Sequence
  Description of Artificial Sequence Synthetic primer
tgatgttccgactatttgttg

SEQ ID NO: 42 - protein, 30 aa, Homo sapiens
GFAFCRECKEAYHEGECSAVFEASGTTTQA

SEQ ID NO: 43 - protein, 30 aa, Pan troglodytes
GFAFCRECKEAYHEGECSALFEASGTTTQA

SEQ ID NO: 44 - protein, 30 aa, Macaca fascicularis; identical to SEQ ID NO: 43

SEQ ID NO: 45 - protein, 30 aa, Rattus norvegicus
GFVFCRDCKEAYHEGECDSMFEASGATSQA

SEQ ID NO: 46 - protein, 30 aa, Mus musculus
GFVFCRDCKEAYHEGDCDSLLEPSGATSQA

SEQ ID NO: 47 - protein, 30 aa, Canis lupus familiaris
GFIFCRECKEEYHEGECSTLFEASGAVTQA

SEQ ID NO: 48 - protein, 13 aa, Homo sapiens
GGRPGLGRGRNSN

SEQ ID NO: 49 - protein, 12 aa, Homo sapiens
KARVALENDDDR

SEQ ID NO: 50 - protein, 10 aa, Homo sapiens
GNKENIKPNE

SEQ ID NO: 51 - protein, 13 aa, Homo sapiens
GGRPGLGRGRNSN

SEQ ID NO: 52 - protein, 13 aa, Homo sapiens
GRRNLLVDAAEAG

SEQ ID NO: 53 - protein, 13 aa, Homo sapiens
VLRKTLDPVFDET

SEQ ID NO: 54 - protein, 12 aa, Homo sapiens
ESRELLQSFDSA

SEQ ID NO: 55 - protein, 13 aa, Homo sapiens
GRRDYLIKIIDDS

SEQ ID NO: 56 - protein, 12 aa, Homo sapiens
CNRAKLFRFDVE

SEQ ID NO: 57 - protein, 12 aa, Homo sapiens
SQRVKLFRFDAE

SEQ ID NO: 58 - protein, 13 aa, Homo sapiens
PVRKNLFRFGEST

SEQ ID NO: 59 - protein, 12 aa, Homo sapiens
SHRAKLYRYDKD

SEQ ID NO: 60 - protein, 13 aa, Homo sapiens
TERVWLWTACDFA

SEQ ID NO: 61 - protein, 13 aa, Homo sapiens
AVRFKLQDVADSF

SEQ ID NO: 62 - protein, 12 aa, Homo sapiens
KERAKLYRWDRD

SEQ ID NO: 63 - protein, 12 aa, Homo sapiens
LVRTVLRAGDRE

SEQ ID NO: 64 - protein, 13 aa, Homo sapiens
VFRNQLPRKNDFY

SEQ ID NO: 65 - protein, 12 aa, Homo sapiens
EAREDLAALEKD

SEQ ID NO: 66 - protein, 12 aa, Homo sapiens
VPRAILVDLEPG

SEQ ID NO: 67 - protein, 12 aa, Homo sapiens
SQRKDLGRSESL

SEQ ID NO: 68 - protein, 12 aa, Homo sapiens
FLRERLTDLEQR

SEQ ID NO: 69 - protein, 13 aa, Homo sapiens
FLRDPLREIEEPY

SEQ ID NO: 70 - protein, 10 aa, Homo sapiens
FSKENSVWNS

SEQ ID NO: 71 - protein, 13 aa, Homo sapiens
GYRNKLAVIGEVL

SEQ ID NO: 72 - protein, 12 aa, Homo sapiens
LLRDDLVLVESP

SEQ ID NO: 73 - protein, 12 aa, Homo sapiens
AQRVLLGLSEPI

SEQ ID NO: 74 - protein, 12 aa, Homo sapiens
SRRALLGYSDQV

SEQ ID NO: 75 - protein, 12 aa, Homo sapiens
VLRSQLDINNKK

SEQ ID NO: 76 - protein, 13 aa, Homo sapiens
LLRKRLPVTNEMV

SEQ ID NO: 77 - protein, 12 aa, Homo sapiens
GSRDKLIQDNRR

SEQ ID NO: 78 - protein, 13 aa, Homo sapiens
DSRLPLILVGEKS

SEQ ID NO: 79 - protein, 12 aa, Homo sapiens
RDRKLLGAEERI

SEQ ID NO: 80 - protein, 12 aa, Homo sapiens
GVRLRLTIVDTR

SEQ ID NO: 81 - protein, 6 aa, Homo sapiens
QMKENY

SEQ ID NO: 82 - protein, 12 aa, Homo sapiens
KDRKLLSAEERI

(19) United States
(12) Patent Application Publication
(10) Pub. No.: US 2020/0323860 A1
(43) Pub. Date: Oct. 15, 2020

(54) METHODS AND MATERIALS FOR TREATING CANCER

(71) Applicant: Mayo Foundation for Medical Education and Research, Rochester, MN (US)

(72) Inventors: Zhenkun Lou, Rochester, MN (US); SeungBaek Lee, Rochester, MN (US); JungJin Kim, Rochester, MN (US)

(73) Assignee: Mayo Foundation for Medical Education and Research, Rochester, MN (US)

(21) Appl. No.: 16/915,719

(22) Filed: Jun. 29, 2020

Related U.S. Application Data
(62) Division of application No. 15/757,744, filed on Mar. 6, 2018.
(60) Provisional application No. 62/215,574, filed on Sep. 8, 2015.

(51) Int. Cl. A61K 31/519; C12Q 1/6886; A61K 31/506; A61P 35/00; A61K 31/192; G01N 33/574

(52) U.S. Cl. CPC A61K 31/519; C12Q 1/6886; A61K 31/506; A61P 35/00; G01N 2800/52; G01N 33/57423; C12Q 2600/106; C12Q 2600/158; A61K 31/192

(57) ABSTRACT

This document provides methods and materials for treating cancer. For example, methods and materials for identifying a mammal as having cancer cells that express little, or no, Parkin mRNA or Parkin polypeptide and administering one or more mitotic kinase inhibitors to treat the mammal identified as having cancer cells with a Parkin deficiency are provided. Methods and materials for identifying a mammal as having a cancer that is responsive to treatment with one or more mitotic kinase inhibitors also are provided.

### CROSS-REFERENCE TO RELATED APPLICATIONS

This application is Divisional of U.S. application Ser. No. 15/757,744, filed Mar. 6, 2018, which is a National Stage application under 35 U.S.C. § 371 of International Application No. PCT/US2016/050761, having an International Filing Date of Sep. 8, 2016, which claims the benefit of U.S. Provisional Ser. No. 62/215,574 filed Sep. 8, 2015. The disclosure of the prior applications is considered part of the disclosure of this application, and are incorporated in their entirety into this application.

### SEQUENCE LISTING

The instant application contains a Sequence Listing which has been submitted electronically in ASCII format and is hereby incorporated by reference in its entirety. Said ASCII copy, created on Jun. 29, 2020, is named 07039-1482002_SL.txt and is 14,929 bytes in size.

### BACKGROUND

### 1. Technical Field

This document relates to methods and materials involved in treating cancer. For example, this document provides methods and materials for using one or more mitotic kinase inhibitors to treat cancers having a Parkin deficiency.

### 2. Background Information

Loss of function of the Parkin protein leads to death of dopaminergic neurons and causes Autosomal Recessive Juvenile Parkinsonism (AR-JP) (Kitada et al., Nature, 392:605-608 (1998); Lucking et al., N. Engl. J. Med., 342:1560-1567 (2000)). Parkin as a RING finger containing protein is capable of promoting mono- and polyubiquitination of target proteins (Moore et al., J. Neurochem., 105:1806-1819 (2008); Olzmann et al., J. Cell. Biol., 178:1025-1038 (2007); and Walden and Martinez-Torres, Cell. Mol. Life Sci., 69:3053-3067 (2012)). The neuroprotective role of Parkin is linked to its role in mitophagy and removal of toxic substrates (Winklhofer, Trends Cell Biol., 24(6):332-341 (2014)). Parkin also has been identified as a candidate tumor suppressor in a wide variety of human cancers (Cesari et al., Proc. Natl. Acad. Sci. USA, 100:5956-5961 (2003); Fujiwara et al., Oncogene, 27:6002-6011 (2008); Picchio et al., Clin. Cancer Res., 10:2720-2724 (2004); Veeriah et al., Nat. Genet., 42:77-82 (2010); and Yeo et al., Cancer Res., 72:2543-2553 (2012)). However, how Parkin functions as a tumor suppressor remains unclear. At the cellular level, loss of Parkin has been associated with formation of micronuclei and multipolar spindles, implying a requirement for Parkin in proper chromosome segregation (Veeriah et al., Nat. Genet., 42:77-82 (2010)). Mechanistically, Cyclin E was proposed as a Parkin substrate contributing to mitotic defects (Veeriah et al., Nat. Genet., 42:77-82 (2010)). However, another group suggested that Cyclin E is not a Parkin substrate (Yeo et al., Cancer Res., 72:2543-2553 (2012)). Therefore, how Parkin regulates mitosis remains unclear.

### SUMMARY

This document provides methods and materials for treating cancer. For example, this document provides methods and materials for identifying a mammal as having cancer cells that express little, or no, Parkin polypeptide and administering one or more mitotic kinase inhibitors to treat the mammal identified as having cancer cells with a Parkin deficiency. As described herein, mammals identified as having cancer cells with a Parkin deficiency can be effectively treated with one or more mitotic kinase inhibitors. This document also provides methods for identifying a mammal as having a cancer that is responsive to treatment with one or more mitotic kinase inhibitors. For example, cancer cells obtained from a mammal having cancer can be assessed to determine if they express little, or no, Parkin mRNA or Parkin polypeptide. If the cancer cells express little, or no, Parkin mRNA or Parkin polypeptide, then the mammal can be classified as having a cancer responsive to treatment with one or more mitotic kinase inhibitors. If the cancer cells do not express little, or no, Parkin mRNA or Parkin polypeptide, then the mammal can be classified as having a cancer that is not responsive to treatment with one or more mitotic kinase inhibitors.

In general, one aspect of this document features a method for treating cancer in a mammal. The method comprises, or consists essentially of, (a) identifying the mammal as having cancer cells that express a reduced level of Parkin, and (b) administering a mitotic kinase inhibitor to the mammal under conditions wherein the number of cancer cells within the mammal is reduced. The mammal can be a human. The cancer can be lung cancer. The cancer cells can express a reduced level of Parkin as compared to the level of Parkin expressed in normal IMR-90 lung fibroblasts, normal WI-38 lung fibroblasts, or normal BES-2B lung immortalized epithelial cells. The mitotic kinase inhibitor can be selected from the group consisting of BI 2536, VX-680, and ON-01910.

In another aspect, this document features a method for identifying a mammal as having cancer susceptible to treatment with a mitotic kinase inhibitor. The method comprises, or consists essentially of, (a) determining that cancer cells of the cancer express a reduced level of Parkin, and (b) classifying the mammal as having cancer susceptible to treatment with the mitotic kinase inhibitor. The mammal can be a human. The cancer can be lung cancer. The cancer cells can express a reduced level of Parkin as compared to the level of Parkin expressed in normal IMR-90 lung fibroblasts, normal WI-38 lung fibroblasts, or normal BES-2B lung immortalized epithelial cells. The mitotic kinase inhibitor can be selected from the group consisting of BI 2536, VX-680, and ON-01910.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIGS. 1A-1E. Parkin Regulates Mitosis. (A) Time-lapse analysis of mitotic U2OS cells transfected with Control or Parkin siRNA. 50 cells were counted in each experiment. Top: Quantification of abnormal mitotic cells. *, p<0.05, **, p<0.01 and ***, p<0.001 versus Control siRNA by one-way ANOVA. Bottom left: Parkin and β-actin expression were shown; Bottom right: Representative images of cells with indicated misaligned chromosome, lagging chromosome, and Chromosome bridge were shown. Scale bar, 10 μm. (B) Cells were synchronized at the G1/S transition by double-thymidine block, and then released into a drug-free medium. Cell were harvested at indicated times and analyzed by immunobloting. p27kip1 serves as a G0-G1 phase marker; Cyclin E, early S phase; Skp2, G1-S; p-H3, mitosis. (C) Subcellular localization of Parkin during each stage of the cell cycle. U2OS cells were stained with antibodies against Parkin (which were red) and Plk1 (which were green) and DNA (which were blue) stained with DAPI. White arrows with tails, centrosome; triangular arrowheads, midzone, midbody, or midring from anaphase to cytokinesis. Scale bar represents 20 μm. (D) Immunoblot analysis of mitotic factors in primary Parkin WT and KO MEFs (Passage 5). (E) Immunoblot analysis of mitotic factors in primary Parkin WT and KO MEFs after releasing from serum starvation (for 72 hours) and nocodazole arrest (for 18 hours). See also, FIGS. 2 and 3.

FIGS. 2A-2K. Suppression of Parkin results in mitotic defects, related to FIG. 1. (A) Live-cell imaging analysis of chromosome segregation errors in mitotic U2OS cells transfected with control or Parkin siRNA. mRFP-H2B positive U2OS cells were captured every 5 minutes by time-lapse fluorescence microscopy. The fluorescence (Top) and phase-contrast (Bottom) images were shown. Numbers indicate the time in minutes after the first frame. Scale bar, 10 μm. (B) Live-cell imaging analysis of chromosome segregation errors in Parkin WT and KO MEFs. Cells in metaphase were analyzed every 3 minutes for 1 hour by time-lapse fluorescence microscopy. Parkin WT (n=105); Parkin KO (n=155). Bar, 10 μm. (C) Parkin expression in (B) was measured by immunoblot. (D and E) Immunofluorescence (D) and FACS analysis (E) of Parkin WT and KO MEFs. (F) Quantification in (B). Quantification of abnormal mitotic cells. *, p<0.05, **, p<0.01 and ***, p<0.001 versus Parkin WT by one-way ANOVA. (G and H) Time-lapse images of mitotic cells in Parkin WT and KO MEFs reconstituted with WT Parkin. (G) Cells were infected with the indicated plasmids, and then cell lysates were blotted with the indicated antibodies. (H) Cells in metaphase were analyzed every 3 minutes for 1 hour by time-lapse fluorescence microscopy. Representative images of chromosome segregation in Parkin WT and KO MEFs reconstituted with Parkin WT retrovirus. Bar, 10 μm. (I) U2OS Cells were synchronized at the G1/S transition by double thymidine block, and then cells were released as in FIG. 1B and analyzed by RT-PCR. (J) Representative confocal images of Parkin's localization during each stage of the cell cycle. U2OS cells were stained with antibodies against Parkin (evident from red stain) and Plk1 (evident from green stain) as a positive marker of centrosome, midzone, midbody or midring during the mitosis, and DNA (evident from blue stain) stained with DAPI. Scale bar represents 20 μm. (K) Quantification in (J).

FIGS. 3A-3F. Parkin regulates mitosis-related proteins in mitosis, related to FIG. 2. (A) Cells were infected with the indicated constructs and arrested with nocodazole. Cells were stained with DAPI (evident from blue stain for DNA), anti-Parkin (evident from green stain), and anti-Plk1 (evident from red stain for centrosome or midbody). White arrow means centrosome in metaphase; multipolar or abnormal cells are indicated by yellow arrows. The scale bar represents 10 μm. (B) Quantification in (A). **, p<0.01 and ***, p<0.001 versus control siRNA by one-way ANOVA. (C) Cells were infected with indicated plasmids, then cells in metaphase were analyzed every 5 minutes by time-lapse fluorescence microscopy. Yellow arrows indicate defective mitotic events. Representative images of mitotic cells. Bar, 10 μm. (D) Extracts collected as in (C) were analyzed by immunoblot. (E) After infection with Control, PINK1, or Parkin shRNA, cells were synchronized by nocodazole treatment. Immunoblots of cell extracts are shown. (F) Parkin assembles K-11 linked polyubiquitin chains on Plk1. HEK 293T cells were transfected with the indicated plasmids (Ubiquitin-chains, WT, K6, K11, K27, K29, K33, K48 and K63 only; GFP-Parkin and Flag-Plk1). Cells were synchronized by nocodazole and treated with MG 132. Ubiquitin conjugates were immunoprecipitated with Flag or HA antibodies and then analyzed by immunoblot.

FIGS. 4A-4D. Parkin-Mediated Regulates the Levels and Ubiquitination of Mitotic Regulators. (A) HEK 293T cells were synchronized by nocodazole for 18 hours, and mitotic and ansynchronized cells were collected for immunoprecipitation (IP)-immunoblot analysis with control IgG, anti-Plk1, Cyclin B1 and Parkin antibodies. (B) HEK 293T cells were transfected with the indicated plasmids, and then treated with MG132 or left untreated. Cell lysates were blotted with the indicated antibodies. (C) The lung, liver, kidney, spleen tissues of Parkin WT and KO mice (n=3 mice/genotype) were lysed, and cell lysates were blotted with the indicated antibodies. (D) HEK 293T cells were transfected with the indicated constructs and arrested in mitosis with nocodazole for 18 hours (Left). Cells were synchronized at the G1/S transition by double-thymidine block, and then released into a new medium (Right). Cells were then treated with MG132. Ubiquitinated proteins were pull down under denaturing conditions by Ni-NTA agarose and analyzed by immunoblot. c-Myc and Cyclin E were shown as negative controls. See also, FIG. 5.

FIGS. 5A-5F. The Protein expression and localization of UbcH7 during mitosis, related to FIG. 2. (A) The same cells as FIG. 1B were analyzed by immunobloting. (B) Cells were infected with the indicated constructs and arrested with nocodazole. Mitotic cells were collected for immunoprecipitation (IP)-immunoblot analysis. Cell lysates were IPed and blotted with the indicated antibodies. (C) UbcH7 localizes to mitotic structures. Cells were stained with anti-UbcH7 (evident from red stain) and anti-Aurora B (evident from green stain) and DAPI to visualize DNA. White arrows point to centrosome from late prophase to cytokinesis, while arrowheads point to midzone and midbody from anaphase to cytokinesis. The scale bar represents 20 μm. (D) Time-lapse analysis of mitotic cells transfected with control or UbcH7 siRNA. Cells were infected with mRFP-H2B. Cells in metaphase were analyzed every 5 minutes for 2 hours by time-lapse fluorescence microscopy. The data represent the average of three experiments, and 60 control or 60 UbcH7 siRNA cells were monitored in each experiment. Quantification of abnormal mitotic cells. *, p<0.05 and **, p<0.01 versus control siRNA by one-way ANOVA (Top table). UbcH7 expression was measured by immunoblot analysis of (left bottom). Lagging chromosome, chromosome bridge, or chromosome-misaligned cells were indicated by yellow arrows. Scale bar, 20 μm (right bottom). (E) Cells were transfected with the indicated constructs and then arrested in mitosis with nocodazole. The cells were stained with DAPI (evident from blue stain), anti-Tubulin (evident from green stain), and anti-Aurora B (evident from red stain). White arrows indicate centrosomes in metaphase; multipolar cells are indicated by yellow arrows; misaligned cell are indicated by pink arrowheads. Scale bar, 20 μm (left). Quantification of (left panel) with protein expression intensity at kinetochore (left graph), multipolar (middle) or abnormal mitotic cells of Aurora B (right) relative to total mitotic cells or normal of Aurora B (in kinetochore) in mitosis results represent the means (±S.E.) of three independent experiments performed in triplicate. **, p<0.01 and ***, p<0.001 by one-way ANOVA. (F) Parkin forms a complex with Cdc20 or Cdh1 as a mitotic regulator. In vitro ubiquitination of Cyclin B1, Securin and Nek2A by Parkin and Cdc20/Cdh1. Purified bacteria-produced His-Cyclin B1, Securin and Nek2A protein was incubated the absence of Ube1, UbcH7, Cdc20, Cdh1, or Parkin as indicated for 90 minutes at 30° C. Samples were analyzed by immunobloting with ubiquitin antibody.

FIGS. 6A-6F. Parkin-Cdc20/Cdh1 Complex Is A Mitotic Regulator during the Cell Cycle. (A) HEK 293T cells were synchronized by nocodazole and treated with MG132. Cell lysates were then subjected to IP and immunoblot as indicated. (B) Purified Cdc20 or Cdh1 were incubated with GST or GST-Parkin coupled to GSH-Sepharose. Proteins retained on Sepharose were then blotted with the indicated antibodies. (C) HEK 293T cells transfected with Flag-tagged WT Parkin were synchronized by nocodazole treatment. Cells were released and subjected to IP and immunoblot with the indicated antibodies. (D and E) Cells were transfected with the indicated constructs and treated as in FIG. 6A. Cells were subjected to IP and immunoblot with the indicated antibodies. APC11 (D) and APC2 (D and E) were shown as negative controls. (F) In vitro ubiquitination of Cyclin B1, Securin and Nek2A by Parkin and Cdc20/Cdh1. Purified bacteria-produced His-Cyclin B1, Securin and Nek2A protein was incubated with different components as indicated for 90 min at 30° C. Samples were analyzed by immunobloting with ubiquitin antibody. See also, FIGS. 5 and 14.

FIGS. 7A-7G. UbcH7-Parkin-Cdc20 and -Cdh1 Complexes Regulate Mitosis Independently of UbcH10-APC/C-Cdc20 and -Cdh1 Complexes. (A-C) Live-cell imaging analysis of chromosome segregation defects in U2OS cells infected with the indicated constructs and synchronized by nocodazole treatment. Cells were fixed and stained with DAPI. Representative images of cells with indicated missegregation events were shown. Scale bar, 20 μm (A). Analysis of numerical chromosome segregation errors. 100 cells were counted in each experiment. *, p<0.05, **, p<0.01 and ***, p<0.001 versus Control shRNA by one-way ANOVA (B-C, Left). Immunoblot analysis with indicated antibodies (B-C, Right). (D) Cells were infected with the indicated constructs, synchronized by nocodazole, and released. Cyclin B1 expression was then examined by immunoblot analysis (Top). FACS analysis for cell cycle profile (Bottom). (E) Fluorescence quantification of Cyclin B1-GFP by time-lapse imaging in mitotic H2B-mRFP-expressing U2OS cells infected with the indicated shRNAs. Cells were plotted against time before and after prometaphase (shake off). *, p<0.05, **, p<0.01 and ***, p<0.001 versus control shRNA by two-way ANOVA. (F) Representative images of cells as indicated. The frames of live cell imaging were recorded by shake-off for mitotic cells. Trypsin-EDTA was the treatment for Interphase cells as the control. Scale bar, 20 μm. (G) After infection with the indicated shRNAs, cells were synchronized at the mitosis transition (prometaphase) by nocodazole treatment for 18 hours. After harvesting the mitotic cells by shake off, cells were re-cultured and dividing cells were examined at the indicated time points. The data represent the average of three experiments, and 100 cells were monitored in each experiment. Scale bar, 20 μm. See also, FIG. 8.

FIGS. 8A-8E. UbcH7-Parkin-Cdc20/Cdh1 complex is distinct from UbcH10-APC/C-Cdc20/Cdh1 complex, related to FIG. 4. (A) Cells were stained with DAPI (evident from blue stain), Parkin (evident from red stain), and APC3 (evident from green stain) antibody. Yellow arrows, centrosome; Pink arrowheads, midzone; White arrows, kinetochore; White arrowheads, midbody; Blue arrows, midring from prophase to cytokinesis. The scale bar represents 20 μm. (B) Summary of localization of endogenous Cdc20, Parkin, and APC3/Cdc27. Blue, Nuclear; Purple, Cytosol; Yellow, Centrosome; White, Kinetochore; Green, Microtubule; Orange, Midzone; Pink, Midbody; Sky blue, Midring; P, Partial signal. (C) Mitotic cells were infected with control, Parkin shRNA, Apc11 shRNA, Cdc20 shRNA. Protein extracts were immunoblotted with the indicated antibodies. (D and E) Cells were transfected with the indicated constructs and collected for FACS and microscopy.

FIGS. 9A-9K. Parkin Is Phosphorylated by Plk1 at Ser378 and Activated during Mitosis. (A) Cells were synchronized at the G1/S transition by double-thymidine block, and cells were released. Cell were harvested at indicated times and analyzed by immunobloting. (B) Cells were incubated in the absence or presence of nocodazole or CCCP and subjected to immunoblot analysis with the indicated antibodies. (C) Comparison of the sequences surrounding 5378 of Parkin orthologues (SEQ ID NOS 42-47, respectively, in order of appearance). (D) Nocodazole-arrested mitotic cells were incubated in the absence or presence of the Plk1 inhibitor (BI 2536) and subjected to IP and immunoblot. Immunoprecipitates were incubated with or without λ phosphatase (PPase) and were analyzed by immunobloting with pS378 antibody. (E) Cells were infected with the indicated constructs, synchronized by nocodazole, and released. Parkin phosphorylation at pS378 was then examined by immunoblot analysis. (F) Cells were transfected with indicated plasmids, and Parkin phosphorylation at pS378 was examined. (G) In vitro kinase assay of Parkin by Plk1. Parkin phosphorylation was visualized by pS378 Parkin antibody. (H) Cells were transfected with the indicated plasmids, and then treated with nocodazole. Cell lysates were then blotted with the indicated antibodies. (I) in vitro ubiquitination of Cyclin B1, Securin and Nek2A by WT Parkin and mutants (S65A, S65D, S378A and S378D). Purified bacteria-produced His-Cyclin B1, Securin and Nek2A protein was incubated with different components as indicated for 90 minutes at 30° C. Samples were analyzed by immunobloting with anti-Cyclin B1, Securin and Nek2A antibody. (J and K) Cells were treated with chemical (J) or transfected with indicated constructs (K). Cells were then collected for IP-immunoblot analysis in the absence or presence of nocodazole. See also, FIG. 10.

FIGS. 10A-10F. Relationship between Parkin and Plk1 protein expression in 400 human non-small cell lung cancers (NSCLC), related to FIG. 5. (A) The domain of Parkin structure (left). Cells were transfected with the indicated plasmids, and mitotic cells were collected for immunoprecipitation (IP)-immunoblot analysis (right). (B) Cells were transfected with indicated constructs and treated with CCCP or nocodazole. Cells were then incubated with MG 132. The ubiquitinated proteins were pulled down under denaturing conditions by Ni-NTA agarose and analyzed by immunoblot. (C-F) Immunohistochemistry showing reciprocal expression of Parkin and Plk1 protein. (C) Representative microscopy images of Parkin (left) and Plk1 (right) in human adenocarcinoma or squamous cell carcinoma compare with normal lung. Serial tumor sections from the same patient were processed. Scale bar, 50 μm. (D) Quantities expression of the Parkin/Plk1 axis in NSCLC for negative correlation. (E) Representative microscopy images of Parkin and Plk1 in NSCLC TMA tissues. Immunostain intensity: 0 (negative), 1+(weak), 2+(moderate), and +3 (strong). (F) Three normal lung cells and seven cell lines (six NSCLCs and one SCLC) were analyzed by immunobloting for the indicated proteins.

FIGS. 11A-11H. Parkin Is A Key Mitotic Regulator Functioning as a Tumor Suppressor. (A) Cells were transfected with the indicated plasmids, and mitotic cells were analyzed by immunoblot for the indicated proteins. (B) Schematic of the experiments. (C) A549 cells stably transfected with doxycycline-inducible constructs encoding WT Parkin or mutant Parkin (S378A and S378D) were treated with doxycycline and subjected to IP and immunoblot as indicated (Top), in vivo ubiquitination (Bottom). (D) Athymic nude mice were injected subcutaneously with A549 cells stably-transfected with vector or doxycycline-inducible Parkin constructs (WT, S378A and S378D). Two days after injection, doxycycline was administered in drinking water. Tumor growth was measured at the indicated times after injection. n=5 for each group. The image shows a representative mouse injected with the indicated cells (Top). Tumor volumes (mm³) were measured at the indicated times after injection (Bottom). *, p<0.05, **, p<0.01 and ***, p<0.001 by two-way ANOVA. (E) Cells were infected with the indicated constructs and were collected for FACS analysis. (F) Parkin WT or KO MEF cells were treated with increasing concentrations of BI 2536 for 3 days, fixed, and stained by 0.2% Crystal violet (Left). Results represent the means (±S.E.) of three experiments performed in triplicate. *, p<0.05 and ***, p<0.001 versus Parkin WT MEFs by one-way ANOVA (Right). (G) Nude mice bearing Parkin WT (Left side) or KO (Right side) MEFs were treated i.v. for four cycles with either the vehicle control (indicated by closed circles or closed squares) or BI 2536 at a dose of 20 mg/kg twice weekly, n=10 per group. Mean transformed MEFs volumes for Parkin KO are shown. ***, p<0.001 and ****, p<0.0001 versus Parkin WT MEFs by two-way ANOVA. (H) Schematic model. See also, FIGS. 12 and 13.

FIGS. 12A-12J. Parkin misregulation is a driving event in tumorigenesis and WT Parkin and S378D have effect to inhibit tumor formation not S378A in Xenograft model, related to FIG. 6. (A-E) Parkin WT and KO MEFs were analyzed for chromosome metaspreading assay (A). Cells were stained with anti-γ-tubulin and DAPI, and numbers of centrosome were counted (B). PDL and passage number of cells (C), SA-β-gal staining for senescence (D), colony formation and soft agar assay (E) were determined. Bioluminescence images of xenografts (left) and immunohistochemistry (right) from MEFs (F). Scale bar, 20 μm (C, D and E). * indicates transformed cells (C). High grade poorly differentiated malignant tumors, 100×, 200× and 400× (F, right). (G) Three NSCLC cells stably transfected with a doxycycline-inducible Parkin construct were treated with doxycycline and immunoblot as indicated. (H) MEFs were subjected to IP and immunoblot as indicated to determine the ubiquitination of Parkin substrates. (I) MEFs were infected with the indicated constructs and were collected for FACS analysis. (J) Athymic nude mice were injected subcutaneously with MEFs cells stably-infected with vector or Parkin constructs (WT, S378A and S378D). Tumor growth was measured at the indicated times after injection. n=5 for each group. The image shows a representative mouse injected with the indicated cells (left). Tumor volumes (mm³) were measured at the indicated times after injection (Right). *, p<0.05, **, p<0.01 and ***, p<0.001 by two-way ANOVA.

FIG. 13A-13F. Transformed and escape senescence events in Parkin KO MEFs were reversed by expressing WT Parkin or downregulation of Plk1 but not C431S or S378A, related to FIGS. 6. (A and B) Parkin WT and KO MEFs under various condition were analyzed for foci formation assay, number of cell growth, SA-β-gal staining for senescence, colony formation assay. Cells were infected or treated with the indicated constructs or chemical and stained (A, left). Cells were infected with the indicated constructs and stained (B, left). Quantification of (left panel) with stained cell intensity results represent the means (±S.E.) of three independent experiments performed in triplicate (right). *, p<0.05, **, p<0.01 and ***, p<0.001 by one-way ANOVA. (C-F) Parkin-deficient lung cancer cell lines but not lung normal fibroblasts were significantly sensitive to Plk1 or Aurora A inhibition by BI 2536 or VX 680. (C and D) Growth inhibition of various lung cancer cell lines by the Plk1 inhibitor, BI 2536 (100 nM) or Aurora A inhibitor, VX-680 (50 nM) for 72 hours. Quantification of cell numbers results represent the means (±S.E.) of three independent experiments performed in triplicate. ***, p<0.001 by one-way ANOVA. (E and F) Growth inhibition of two lung cancer cell lines and normal fibroblasts by the Pk1 inhibitor, BI 2536 in a dose dependent manner for 3 days.

FIG. 14. Alignment of a canonical degradation box (D-box) motif and KEN box in 14 known Parkin substrates, related to FIG. 3 (SEQ ID NOS 1, 48-82, respectively, in order of appearance). All sequences were taken from GenBank® (human origin). The alignment includes the 14 D-box and three KEN box sequence alignments on which the Parkin's substrates domain designation is based. Identical residues have a red color and yellow box; D-box (RXXLXXXXN/D/E, RXXLXXXN/D/E), red color and pink box; KEN box (KENXXXN (SEQ ID NO: 1)).

### DETAILED DESCRIPTION

This document provides methods and materials for treating cancer. For example, this document provides methods and materials for identifying a mammal as having cancer cells that express little, or no, Parkin mRNA or Parkin polypeptide and administering one or more mitotic kinase inhibitors to treat the mammal identified as having cancer cells with a Parkin deficiency. Any appropriate mammal having cancer can be treated as described herein. For example, humans and other primates such as monkeys having cancer can be identified as having cancer cells with a Parkin deficiency and treated with one or more mitotic kinase inhibitors to reduce the number of cancer cells present within the human or other primate. In some cases, dogs, cats, horses, cows, pigs, sheep, mice, and rats can be identified and treated with one or more mitotic kinase inhibitors as described herein.

Any appropriate cancer can be assessed for a Parkin deficiency and, if present, treated as described herein. For example, breast cancer, ovarian cancer, osteosarcoma, lung cancer, prostate cancer, liver cancer, pancreatic cancer, brain/CNS tumors, colon cancer, rectal cancer, colorectal cancer, cervical cancer, or melanoma can be assessed for reduced Parkin expression and treated with one or more one or more mitotic kinase inhibitors as described herein.

Any appropriate method can be used to identify a mammal having cancer. For example, imaging techniques and biopsy techniques can be used to identify mammals (e.g., humans) having cancer.

Once identified as having cancer, the cancer can be assessed to determine if the cancer cells express a reduced level of Parkin. Any appropriate method can be used to identify cancer cells as having a reduced level of Parkin. For example, mRNA-based assays such as RT-PCR can be used to identify cancer cells as expressing little, or no, Parkin mRNA. In some cases, polypeptide-based assays such as antibody staining techniques or ELISAs using anti-Parkin antibodies can be performed to identify cancer cells as expressing little, or no, Parkin polypeptide.

Once identified as having cancer cells with a reduced level of Parkin expression, the mammal can be administered or instructed to self-administer one or more mitotic kinase inhibitors to reduce the number of cancer cells present within the mammal. Examples of mitotic kinase inhibitors include, without limitation, BI 2536 ((R)-4-(8-cyclopentyl-7-ethyl-5-methyl-6-oxo-5,6,7,8-tetrahydropteridin-2-ylamino)-3-methoxy-N-(1-methylpiperidin-4-yl)benzamide), VX-680 (N-(4-(4-(5-methyl-1H-pyrazol-3-ylamino)-6-(4-methylpiperazin-1-yl)pyrimidin-2-ylthio)phenyl)-cyclopropanecarboxamide), and ON-01910 (N-[2-methoxy-5-[[[2-(2,4,6-trimethoxyphenyl)ethenyl]sulfonyl]methyl]phenyl]-glycine, sodium salt (1:1)). In some cases, two or more mitotic kinase inhibitors (e.g., two, three, four, five, or more mitotic kinase inhibitors) can be administered to a mammal to reduce the number of cancer cells present within the mammal.

In some cases, one or more mitotic kinase inhibitors can be administered to a mammal once or multiple times over a period of time ranging from days to weeks. In some cases, one or more mitotic kinase inhibitors can be formulated into a pharmaceutically acceptable composition for administration to a mammal having cancer. For example, a therapeutically effective amount of a mitotic kinase inhibitor (e.g., BI 2536, VX-680, or ON-01910) can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules.

Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A pharmaceutical composition containing one or more mitotic kinase inhibitors can be designed for oral or parenteral (including subcutaneous, intramuscular, intravenous, and intradermal) administration. When being administered orally, a pharmaceutical composition can be in the form of a pill, tablet, or capsule. Compositions suitable for parenteral administration include aqueous and non-aqueous sterile injection solutions that can contain anti-oxidants, buffers, bacteriostats, and solutes that render the formulation isotonic with the blood of the intended recipient. The formulations can be presented in unit-dose or multi-dose containers, for example, sealed ampules and vials, and may be stored in a freeze dried (lyophilized) condition requiring only the addition of the sterile liquid carrier, for example, water for injections, immediately prior to use. Extemporaneous injection solutions and suspensions may be prepared from sterile powders, granules, and tablets.

In some cases, a pharmaceutically acceptable composition including one or more mitotic kinase inhibitors can be administered locally or systemically. For example, a composition provided herein can be administered locally by injection into tumors. In some cases, a composition provided herein can be administered systemically, orally, or by injection to a mammal (e.g., a human).

Effective doses can vary depending on the severity of the cancer, the route of administration, the age and general health condition of the subject, excipient usage, the possibility of co-usage with other therapeutic treatments such as use of other agents, and the judgment of the treating physician.

An effective amount of a composition containing one or more mitotic kinase inhibitors can be any amount that reduces the number of cancer cells present within the mammal without producing significant toxicity to the mammal. For example, an effective amount of a mitotic kinase inhibitor such as ON-01910 can be from about 50 mg/m² to about 2400 mg/m². In some cases, between about 70 mg and about 560 mg of a mitotic kinase inhibitor can be administered to an average sized human (e.g., about 75-85 kg human) daily for about 2 to about 4 weeks.

If a particular mammal fails to respond to a particular amount, then the amount of a mitotic kinase inhibitor can be increased by, for example, two fold. After receiving this higher amount, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition (e.g., cancer) may require an increase or decrease in the actual effective amount administered.

The frequency of administration of a mitotic kinase inhibitor can be any amount that reduces the number of cancer cells present within the mammal without producing significant toxicity to the mammal. For example, the frequency of administration of a mitotic kinase inhibitor can be from about two to about three times a week to about two to about three times a month. The frequency of administration of a mitotic kinase inhibitor can remain constant or can be variable during the duration of treatment. A course of treatment with a composition containing a mitotic kinase inhibitor can include rest periods. For example, a composition containing one or more mitotic kinase inhibitors can be administered daily over a two week period followed by a two week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., cancer) may require an increase or decrease in administration frequency.

An effective duration for administering a composition containing one or more mitotic kinase inhibitors can be any duration that reduces the number of cancer cells present within the mammal without producing significant toxicity to the mammal. In some cases, the effective duration can vary from several days to several weeks. In general, the effective duration for reducing the number of cancer cells present within the mammal can range in duration from about one week to about four weeks. Multiple factors can influence the actual effective duration used for a particular treatment. For example, an effective duration can vary with the frequency of administration, effective amount, use of multiple treatment agents, route of administration, and severity of the condition being treated.

In certain instances, a course of treatment, the number of cancer cells present within a mammal, and/or the severity of one or more symptoms related to the condition being treated (e.g., cancer) can be monitored. Any appropriate method can be used to determine whether or not the number of cancer cells present within a mammal is reduced. For example, imaging techniques can be used to assess the number of cancer cells present within a mammal.

The invention will be further described in the following examples, which do not limit the scope of the invention described in the claims.

### EXAMPLES

### Example 1—Parkin Regulates Mitosis and Genomic Stability Through Cdc20/Cdh1

### Mouse Strains and MEFs

Mouse strains were described elsewhere (Goldberg et al., J. Biol. Chem., 278:43628-43635 (2003)). Parkin (E5355) clone 1 and 8 WT MEFs and Parkin (E5314) clone 1 and 2 KO MEFs were obtained from Dr. Jie Shen (Center for Neurologic Diseases, Harvard Medical School, Brigham and Women's Hospital, Boston, Mass.) and were described elsewhere (Goldberg et al., J. Biol. Chem., 278:43628-43635 (2003)). Parkin KO C57BL/6 (6-8 weeks old, female) mice were purchased from the Jackson Laboratory (Bar Harbor, Me., USA) and mated. Mouse embryonic fibroblasts were isolated from embryonic day 11.5-13.5 (E11.5-E13.5) by uterine dissection for individual embryos. Each embryo was washed softly with 1×PBS (pH 7.2), followed by removal of the mouse embryo's head and liver. The embryo body was suspended in 0.5 mL of 0.25% Trypsin-EDTA, and then forced through a 1 mL syringe with an 18-gauge needle. The tissue homogenate was incubated for 30 minutes at 37° C., triturated by drawing the suspension through a pipette, and then evenly-divided into two 10 cm tissue culture dishes in Dulbecco's modified Eagle's medium (DMEM) with 15% fetal bovine serum (FBS). Early-passage MEFs (passage 1-5) were used for all experiments, and at least three lines were examined for all studies. Animals were housed in a pathogen-free barrier environment throughout the study.

### Cells and Cell Lines and Reagents

All cell lines were sourced from commercial venders. Human embryonic kidney (HEK) 293T, human osteosarcoma U205, HeLa cervix carcinoma cells were cultured in Dulbecco's modified Eagle's media (DMEM, Gibco-Invitrogen). Three normal lung (2 fibroblasts, IMR-90 and WI-38; 1 epithelial cells; BEAS-2B) cells, six NSCLCs (4 adenocarcinoma, H1437, H522, H1650 and A549; 2 large cell carcinoma, H460 and H1299), and one SCLC (H196) cells were maintained in Eagle's minimal essential media (EMEM, Gibco-Invitrogen, Grand Island, N.Y.). The human lung fibroblast IMR-90 and WI-38 cells were obtained from the American Type Culture Collection (ATCC, Manassas, Va.), and cells ranging from 29 to 34 in population doubling level (PDL) were used. These cells were cultured in Eagle's minimal essential media (EMEM, Gibco-Invitrogen, Grand Island, N.Y.). All media contained 10% (15%; IMR-90 and WI-38 cells) heat-inactivated FBS (Gibco-Invitrogen), sodium bicarbonate (2 mg/mL; Sigma-Aldrich, St Louis, Mo.), penicillin (100 units/mL), and streptomycin (100 μg/mL; Gibco-Invitrogen). N-carbobenzoxy-1-leucinyl-lleucinyl-1-norleucinal (MG 132) was purchased from Sigma-Aldrich. BI 2536 and VX-680 were obtained from Selleckchem (Houston, Tex.).

### Plasmids

HA or Flag-tagged Parkin (empty and WT), GFP-tagged Parkin (empty and WT) were obtained from Dr. Jennifer L. B. Roshek, Dr. Darren J. Moore, and Dr. Ted M. Dawson (The Johns Hopkins University School of Medicine, Baltimore, Md.) and Dr. Erkang Fei and Dr. Guanghui Wang (University of Science & Technology of China, China) and were described elsewhere (Moore et al., J. Neurochem., 105:1806-1819 (2008); Rothfuss et al., Hum. Mol. Genet., 18:3832-3850 (2009); and Chen et al., J. Biol. Chem., 285:38214-38223 (2010)). HA or GFP-tagged Parkin (empty and WT, S65A, S65D, S378A, S378D, C431A and C431S) were obtained from Dr. Noriyuki Matsuda (Tokyo Metropolitan Institute of Medical Science, Tokyo, Japan) and were described elsewhere (Iguchi et al., J. Biol. Chem., 288:22019-22032 (2013)). Myc-tagged Parkin (empty and WT, S101A, S131A, S136A, S296A, S378A, S384A and S407A) were obtained from Dr. Christian Haass (Laboratory of Alzheimer's and Parkinson's Disease Research, Department of Metabolic Biochemistry, Ludwig Maximilians University, Germany) (Yamamoto et al., J. Biol. Chem., 280:3390-3399 (2005)). For doxycycline-inducible Parkin constructs, the pcDNA6/TR-Parkin was obtained from Dr. Nadja Patenge (Center of Neurology and Hertie Institute for Clinical Brain Research, Tübingen, Germany) and was described elsewhere (Rothfuss et al., Hum. Mol. Genet., 18:3832-3850 (2009)). pGEX-4T1-Plk1 was obtained from Dr. Ingrid Hoffmann (Cell Cycle Control and Carcinogenesis, German Cancer Research Center) and was described elsewhere (Zhu et al., J. Cell Biol., 200:773-787 (2013)). Myc-tagged Nek2A (Vector, W T and del-KEN box) was obtained from Dr. Andrew M. Fry (Department of Biochemistry, University of Leicester) and was described elsewhere (Hames et al., Biochem. J., 361:77-85 (2001)). Human Myc-tagged Cyclin B1 (WT and del D-box) and Human Myc-tagged Securin (WT, and D-box mutant) constructs were obtained from Dr. Hongtao Yu and Ross Warrington (Howard Hughes Medical Institute, University of Texas Southwestern Medical Center) and was described elsewhere (Tian et al., PNAS, 109:18419-18424 (2012)). The pMX retroviral vector containing the human cDNAs for HA-Parkin Plasmids encoding HA-tagged ubiquitin and ubiquitin lysine mutants, such as K-6 only, K-11 only, K-27 only, K-29 only, K-33 only, K-48 only and K-63 only working, were obtained from Addgene.

### Time-Lapse Live Microscopy

For mitotic timing experiments, mRFP-H2B stably expressing U2OS cells were transfected or infected with control, Parkin, UbcH7, APCJJ, Parkin+APC11, or Cdc20 shRNA (or siRNA). For chromosome missegregation analysis, mRFP-H2B positive Parkin WT or KO MEFs were followed at interframe intervals of 3 or 5 minutes as described elsewhere (van Ree et al., J. Cell Biol., 188:83-100 (2010)). MEFs were seeded onto 35-mm glass bottom dishes (MatTek Corporation). All experiments were performed using a microscope system (Axio Observer; Carl Zeiss MicroImaging, Inc.) with CO₂ Module S, TempModule S, Heating Unit XL S, a plan Apo 63× NA 1.4 oil differential interference contrast III objective (Carl Zeiss MicroImaging, Inc.), camera (AxioCam MRm; Carl Zeiss MicroImaging, Inc.), and AxioVision 4.6 software (Carl Zeiss MicroImaging, Inc.). Imaging medium was kept at 37° C. The mRFP-H2B was obtained from Dr. Jan M. van Deursen. Prism software (for Mac; version 4.0 a; GraphPad Software, Inc.) was used for statistical analysis. At least three independent clones per genotype were used in the aforementioned experiments unless otherwise noted.

### Cell Synchronizations

To synchronize, HeLa cells were treated with 2.5 mM thymidine for 16 hours, released for 8 hours into fresh new 10% serum media, and then treated again with thymidine for 16 hours. After rinsing three times with phosphate-buffered saline (PBS) for 5 minutes, cells were cultured for different times as indicated in each experiment. The cell lysates were harvested and analyzed by immunoblot analysis. For phase marker indication, p27^{kip1} was used as a G₀-G₁ phase marker, Cyclin E was used as early S phase marker, Skp2 p45 was used as a G₁-S marker, and \textcircled{P}-H3 was used as a mitosis marker.

### FACS Analysis

DNA content was measured following staining of cells with propidium iodide. Cells were subsequently trypsinized, washed once in cold PBS, and fixed in 70% ethanol at −20° C. overnight. Fixed cells were pelleted and stained in propidium iodide solution (50 μg/mL propidium iodide, 50 μg/mL RNase A, 0.1% Triton X-100, and 0.1 mM EDTA) in the dark at 4° C. for 1 hour prior to flow cytometric quantification of DNA by a FACScan (Becton Dickinson).

Gene Silencing by siRNAs and Lentiviral shRNAs

Parkin, APC11, Cdc20, UbcH7, UbcH10, Plk1 and PINK1 were obtained from Sigma-Aldrich and Open Biosystems.

Pakin shRNA

| Com- | | Clone | | Target sequence |
|---|---|---|---|---|
| pany | Species | Set ID | Names | (5' - - 3') |
| Open | Human | NM_013988 | 84517 | 5'-GAGAGAGTTCTCACA |
| Bio. | | | | TTTAAT-3' |
| | | | | (SEQ ID NO: 2) |
| Open | Human | NM_013988 | 84518 | 5'-ACTCACTAGAATATT |
| Bio. | | | | CCTTAT-3' |
| | | | | (SEQ ID NO: 3) |
| Open | Human | NM_013988 | 84520 | 5'-GAACGTTTAGAAATG |
| Bio. | | | | ATTTCAAA-3' |
| | | | | (SEQ ID NO: 4) |

Pakin shRNA

| | | Clone | | Target sequence |
|---|---|---|---|---|
| Company | Species | Set ID | Names | (5' - - 3') |
| Sigma | Human | NM_013988 | 2399 | 5'-CGTGAACATAACT |
| (TRC1) | | | | GAGGGCAT-3' |
| | | | | (SEQ ID NO: 5) |
| Sigma | Human | NM_013988 | 341 | 5'-CGCAACAAATAGT |
| (TRC1) | | | | CGGAACAT-3' |
| | | | | (SEQ ID NO: 6) |
| Sigma | Human | NM_013988 | 425 | 5'-CGTGATTTGCTTA |
| (TRC1) | | | | GACTGTTT-3' |
| | | | | (SEQ ID NO: 7) |
| Sigma | Human | NM_013988 | 434 | 5'-CTTAGACTGTTTC |
| (TRC1) | | | | CACTTATA-3' |
| | | | | (SEQ ID NO: 8) |
| Sigma | Human | NM_013988 | 872 | 5'-CTCCAAAGAAACC |
| (TRC1) | | | | ATCAAGAA-3' |
| | | | | (SEQ ID NO: 9) |

* Used shRNA-341 and 434

Cdc20 shRNA

| | | Clone | | Target sequence |
|---|---|---|---|---|
| Company | Species | Set ID | Names | (5' - - 3') |
| Sigma | Human | NM_001255 | 1079 | 5'-TGGTGGTAATGAT |
| (TRC2) | | | | AACTTGGT-3' |
| | | | | (SEQ ID NO: 10) |
| Sigma | Human | NM_001255 | 1602 | 5'-AGACCAACCCATC |
| (TRC2) | | | | ACCTCAGT-3' |
| | | | | (SEQ ID NO: 11) |
| Sigma | Human | NM_001255 | 631 | 5'-ATGCGCCTGAAAT |
| (TRC2) | | | | CCGAAATG-3' |
| | | | | (SEQ ID NO: 12) |
| Sigma | Human | NM_001255 | 872 | 5'-GCAGAAACGGCTT |
| (TRC2) | | | | CGAAATAT-3' |
| | | | | (SEQ ID NO: 13) |
| Sigma | Human | NM_001255 | 921 | 5'-CTAAGCTGGAACA |
| (TRC2) | | | | GCTATATC |
| | | | | (SEQ ID NO: 14) |

* Used shRNA-1079 and 872

UbcH7 shRNA

| | | Clone | | Target sequence |
|---|---|---|---|---|
| Company | Species | Set ID | Names | (5' - - 3') |
| Sigma | Human | NM_0033347 | 249 | 5'-CCAGCAGAGT |
| (TRC2) | | | | ACCCATTCAAA-3' |
| | | | | (SEQ ID NO: 15) |
| Sigma | Human | NM_0033347 | 270 | 5'-CCACCGAAGA |
| (TRC2) | | | | TCACATTTAAA-3' |
| | | | | (SEQID NO: 16) |
| Sigma | Human | NM_0033347 | 328 | 5'-AGGTCTGTCT |
| (TRC2) | | | | GCCAGTAATTA-3' |
| | | | | (SEQ ID NO: 17) |
| Sigma | Human | NM_0033347 | 459 | 5'-GAATACTCTA |
| (TRC2) | | | | AGGACCGTAAA-3' |
| | | | | (SEQ ID NO: 18) |
| Sigma | Human | NM_0033347 | 918 | 5'-CACTTTCTGG |
| (TRC2) | | | | CACCGAGTTTA-3' |
| | | | | (SEQ ID NO: 19) |

* Used shRNA-270 and 459

UbcH10 shRNA

| | | Clone | | Target sequence |
|---|---|---|---|---|
| Company | Species | Set ID | Names | (5' - - 3') |
| Sigma | Human | NM_007019 | 290 | 5'-TGGAACAGTA |
| (TRC2) | | | | TATGAAGACCT-3' |
| | | | | (SEQ ID NO: 20) |
| Sigma | Human | NM_007019 | 347 | 5'-CCCTTACAAT |
| (TRC2) | | | | GCGCCCACAGT-3' |
| | | | | (SEQ ID NO: 21) |
| Sigma | Human | NM_007019 | 454 | 5'-TGTATGATGT |
| (TRC2) | | | | CAGGACCATTC-3' |
| | | | | (SEQ ID NO: 22) |
| Sigma | Human | NM_007019 | 575 | 5'-CCTGCAAGAA |
| (TRC2) | | | | ACCTACTCAAA-3' |
| | | | | (SEQ ID NO: 23) |
| Sigma | Human | NM_007019 | 634 | 5'-GCCTGTCCTTG |
| (TRC2) | | | | TGTCGTCTTT-3' |
| | | | | (SEQ ID NO: 24) |

* Used shRNA-454 and 575

APC11 shRNA

| | Spe- | Clone | | Target sequence |
|---|---|---|---|---|
| Company | cies | Set ID | Names | (5' - - 3') |
| Sigma | Human | NM_016476 | 202 | 5'-CAACGATGAGA |
| (TRC1.5) | | | | ACTGTGGCAT-3' |
| | | | | (SEQ ID NO: 25) |
| Sigma | Human | NM_016476 | 225 | 5'-GCAGGATGGCA |
| (TRC1.5) | | | | TTTAACGGAT-3' |
| | | | | (SEQ ID NO: 26) |
| Sigma | Human | NM_016476 | 313 | 5'-CCACATGCATT |
| (TRC1.5) | | | | GCATCCTCAA-3' |
| | | | | (SEQ ID NO: 27) |
| Sigma | Human | NM_016476 | 376 | 5'-CCGCCAGGAAT |
| (TRC1.5) | | | | GGAAGTTCAA-3' |
| | | | | (SEQ ID NO: 28) |
| Sigma | Human | NM_016476 | 489 | 5'-GCTGCAACAAG |
| (TRC1.5) | | | | GTGGAAACAA-3' |
| | | | | (SEQ ID NO: 29) |

* Used shRNA-225 and 376

Plk1 shRNA

| | | Clone | | Target sequence |
|---|---|---|---|---|
| Company | Species | Set ID | Names | (5' - - 3') |
| Sigma | Mouse | NM_011121 | 1484 | 5'-CCTCTCAAA |
| (TRC1.5) | | | | GTCCTCAATAAA-3' |
| | | | | (SEQ ID NO: 30) |
| Sigma | Mouse | NM_011121 | 1903 | 5'-CCTCAACTAT |
| (TRC1.5) | | | | TTCCGCAATTA-3' |
| | | | | (SEQ ID NO: 31) |

PINK1 shRNA

| | | Clone | | Target sequence |
|---|---|---|---|---|
| Company | Species | Set ID | Names | (5'- -3') |
| Open | Human | NM_032409 | 234804 | 5'-CGTATGTGCCT |
| Bio. | | | | TGAACTGAATTAGT |
| | | | | GAAGCCACAGATGT |
| | | | | AATTCAGTTCAAGG |
| | | | | CACATACGT-3' |
| | | | | (SEQ ID NO: 32) |
| Open | Human | NM_032409 | 235108 | 5'-GGGAGCCATCGC |
| Bio. | | | | CTATGAAATTAGTGA |
| | | | | AGCCACAGATGTAAT |
| | | | | TTCATAGGCGATGGC |
| | | | | TCCCA-3' |
| | | | | (SEQ ID NO: 33) |
| Open | Human | NM_032409 | 238759 | 5'-GCCGCAAATGTG |
| Bio. | | | | CTTCATCTATAGTGA |
| | | | | AGCCACAGATGTATA |
| | | | | GATGAAGCACATTTG |
| | | | | CGGCT-3' |
| | | | | (SEQ ID NO: 34) |

* Used shRNA-234804 and 238759

| | |
|---|---|
| Parkin | (1) 5'-GCUUAGACUGUUUCCACUU-3' |
| siRNA | (SEQ ID NO: 35) (sense strand) |
| and | |
| | (2) 5'-CGUGAACAUAACUGAGGGCAU-3' |
| | (SEQ ID NO: 36) (sense strand) |
| UbcH7 | (1) 5'-AAAUGUGGGAUGAAAAACUUC-3' |
| siRNA | (SEQ ID NO: 37) (sense strand) |
| and | |
| | (2) 5'-AGGUCUGUCUGCCAGUAAUUA-3' |
| | (SEQ ID NO: 38) (sense strand) |
| Control | 5'-UUCAAUAAAUUCUUGAGGU-3' |
| siRNA | (SEQ ID NO: 39) (sense strand) |

Reverse Transcription (RT)-PCR of cDNA

RNA preparation, cDNA, and RT-PCR were performed as described elsewhere (Lee et al., J. Cell Sci., 124:1911-1924 (2011)). The following primers were used: The Parkin Forward primer sequence was 5′-CCAG-TGACCATGATAGTGTT-3′ (SEQ ID NO: 40), Reverse primer sequence was 5′-TGATGTTCCGAC-TATTTGTTG-3′ (SEQ ID NO: 41), and β-actin sequence were described elsewhere (Lee et al., J. Cell Sci., 124:1911-1924 (2011)).

### Co-Immunoprecipitation, Immunobloting, and Antibodies

For immunoprecipitation, extraction of proteins with a modified buffer from cultured cells was followed by immunoprecipitation and immunobloting with corresponding antibodies. Rabbit polyclonal antibodies recognizing Parkin (ab15954; the antibody used for most of data), Parkin pS378 (ab65933), Aurora A (ab12875), Aurora B (ab2254), UbcH10 (ab12290), Securin (ab26273), APC11 (ab44708), PINK1 (ab23707), Cyclin E (ab7959) were obtained from Abcam. Mouse monoclonal antibodies recognizing Aurora A (ab13824), Cdh1 (ab3242), APC2 (ab123855), APC11 (ab57158), and c-Myc (ab32072) were purchased from Abcam. Rabbit polyclonal antibody recognizing Aurora B (sc-25426), Mad2 (sc-28261) and Tom20 (sc-11415) were obtained from Santa Cruz Biotechnology. Mouse monoclonal antibody recognizing Parkin (sc-32282), Cyclin E (sc-247), Cyclin B1 (sc-245), and Cdc20 (sc-5296) were purchased from Santa Cruz Biotechnology. Mouse monoclonal antibody recognizing p27^{kip1}, UbcH7, (610853) and APC3 (610455) were obtained from BD transduction Laboratories. Mouse monoclonal antibody recognizing Parkin (#4211S) was obtained from Cell Signaling. Rabbit polyclonal antibody recognizing Parkin (#2132S) was purchased from Cell Signaling. Mouse monoclonal antibody recognizing Plk1 was obtained from Invitrogen. Rabbit polyclonal antibody recognizing Skp2 (NBP1-30077) was obtained from Novus Biologicals. Anti-α-tubulin, Myc, FLAG (m2), and HA mouse antibodies were purchased from Sigma. Rabbit polyclonal homemade antibody recognizing Mad1, Mad2, Bub1, Bub3, Securin, BubR1, and \textcircled{P}-H3 were obtained from Dr. Jan M. van Deursen.

For removing heavy chain, light-chain-specific anti-mouse and anti-rabbit IgG secondary antibodies were obtained from Jackson Immunoresearch and used. For in vivo ubiquitination assays, cells were lysed by urea lysis buffer (8 M urea, 0.1 M Na₂HPO₄, 0.1 M Tris/HCl (pH 8.0), 0.05% Tween 20 and 0.01 M imidazole). After centrifugation, the supernatants were collected and incubated with 20 mL Ni-NTA agarose beads (Qiagen) for four hours at 4° C. The precipitates were washed three times with urea wash buffer (8 M urea, 0.1 M Na₂HPO₄, 0.1 M Tris/HCl (pH 8.0), 0.05% Tween 20, and 0.02 M imidazole) and native wash buffer (0.1 M Na₂HPO₄, 0.1 M Tris/HCl (pH 8.0), 0.05% Tween 20 and 0.02 M imidazole), and were boiled with SDS loading buffer, and then subjected to SDS-PAGE followed by immunoblot analysis.

### Expression and Purification of the Recombinant Protein

HA or GFP-tagged Parkin (empty and WT, S65A, S65D, S378A and S378D) obtained from Dr. Noriyuki Matsuda (Tokyo Metropolitan Institute of Medical Science, Tokyo, Japan) also was cloned into pGEX-4T-1 (Amersham Pharmacia Biotech, Piscataway, N.J.) vector using EcoRI/NotI restriction enzyme sites as described elsewhere (Yamamoto et al., J. Biol. Chem., 280:3390-3399 (2005)). BL21 E. coli (Life Technologies) expressing was transformed with the pGEX-4T-1 (GST-only, WT, S65A, S65D, S378A and S378D) vectors. Positive E. coli BL21 colonies, containing pGEX-4T-1/Parkin, were cultured in 3-5 mL Luria-Bertani (LB) solid medium (with ampicillin) at 37° C. overnight, after which the culture was transferred to fresh 600 mL LB liquid medium (with ampicillin) for 2-3 hours. When the optical density reached a wavelength of 400-600 nm, isopropyl β-D-1-thiogalactopranoside (IPTG) was added with a final concentration of 0.4 M, and the culture was shaken at 18° C. overnight. The bacteria were then collected, and then sonicated on ice in 1×NETN buffer supplemented with complete protease inhibitor, aprotinin. After centrifugation at 5,000×g for 10 minutes at 4° C., the supernatant was purified using a glutathione S-transferase (GST) purification resin column (Novagen; Merck KGaA, Darmstadt, Germany) including with aprotinin and PMSF for 18 hours with rocking at 4° C., according to the manufacturer's instructions. After six washes with 1×NETN, GST-Parkin was eluted with GSH elution buffer (30 mM reduced glutathione, 1% Triton X-100, 500 mM Tris-HCl, pH 8.8). The integrity and yield of purified GST fusion proteins, as well as commercial Cdc20 (Novus Biologicals, H00000991-P01) and Cdh1 recombinant proteins (Novus Biologicals, H00051343-P01) were assessed by SDS PAGE followed by Coomassie blue staining. All His-tagged recombinant proteins were purified using TALON resin (CLONTECH) according to the manufacturer's protocol with minor modifications. Beads were washed three times with 10 mL of PB buffer (200 mM washing buffer). Proteins were eluted with 300-500 mL of elution buffer (same as binding buffer except with 100 mM imidazole). Eluted proteins were concentrated to 1-2 mg per mL using a microconcentrator (Filtron). Protein samples were fractionated on 10% SDS polyacrylamide gels and stained by Coomassie brilliant blue G250.

### In Vivo and In Vitro Ubiquitination Assays

For in vivo ubiquitination, cells were transfected with ubiquitin-his plasmid together with HA or HA-Parkin (WT, C431S) followed by treatment with MG 132 (10 μM). 48 hours post-transfection, cells were lysed by Urea lysis buffer (8 M Urea, 0.1 M Na₂HPO₄, 0.1 M Tris/HCl (pH 8.0), 0.05% Tween 20 and 0.01 M imidazole). After centrifugation, the supernatants were collected and incubated with 20 mL Ni-NTA agarose beads (Quiagen) for 4 hours at 4° C. The precipitates were washed three times with Urea wash buffer (8 M Urea, 0.1 M Na₂HPO₄, 0.1 M Tris/HCl (pH 8.0), 0.05% Tween 20, and 0.02 M imidazole) and Native wash buffer (0.1 M Na₂HPO₄, 0.1 M Tris/HCl (pH 8.0), 0.05% Tween 20, and 0.02 M imidazole), and were boiled with SDS loading buffer, and then subjected to SDS-PAGE followed by immunoblot analysis. In vitro ubiquitination assay was performed in 300_, of ubiquitination reaction buffer (50 mM Tris⋅HCl pH 7.5, 2 mM MgCl₂, 2 mM ATP, 10 μg/μL Myc-ubiquitin), 50 ng of E1 (Ube1; Boston Biochem), 200 ng of E2 (UbcH7; Boston Biochem), 2 μg of E3 (purified Parkin, Wt, S65A, S65D, S378A and S378D), and 10 ng of cofactor (Cdh1 or Cdc20; Abnova). Parkin, Nek2A, Securin, and Cyclin B1 was cloned into pGEX-2TK, pGEX-4T-1 or pRSETA and were purified. The reaction was performed for 90 minutes at 30° C. Equal volumes of each sample were prepared for immunoblot. The reaction products were analyzed by immunoblot with ubiquitin antibody.

### In Vivo Kinase Assays

For in vivo kinase assays, GST or GST-Parkin (WT, S378A) purified recombinant proteins were incubated with active baculovirus-expressed human Plk1 in kinase buffer. The kinase assays were carried out in 30 μL reaction, containing 50 mM Tris-HCl, 10 mM MgCl₂, 2 mM DTT, 1 mM EGTA, 0.01% Brij (pH 7.5), 50 mM cold ATP, 50 ng Plk1, and purified recombinant proteins. The reactions were incubated at 30° C. for 30 minutes, and immunoblotted with indicated antibodies.

### Immunofluorescence and Confocal Microscopy

For immunofluorescence staining, HeLa, MEF, or IMR-90 cells were plated on glass coverslips and transfected with the indicated constructs. Cells were then fixed in 3.7% paraformaldehyde for 10 minutes at room temperature and stained using standard protocols. Immunofluorescence images were taken using fluorescent microscopy (Nikon Microscope, Melville, N.Y.). For confocal microscopy, fluorescence images were obtained by A laser-scanning microscope (LSM 510 v3.2SP2; Carl Zeiss) and equipped with a microscope (Axiovert 100 M, Carl Zeiss) with a c-Apochromat 100× oil immersion objective was used to analyze immune-stained cells and to capture representative images.

### In Vitro Binding Assay

GST fusion proteins were prepared following standard protocol. For in vitro biding assays, Parkin GST fusion proteins bounds to the GSH sepharose were incubated with cell lysates. After washing, the bound proteins were separated by SDS-PAGE and immunoblotted with indicated antibodies.

### Colony Formation or Foci Assay, Senescence-Associated β-Galactosidase (Gal) Staining

For colony formation or foci assay, early-passage MEFs (passage 5) cells were plated at low density into 60-mm cell culture plates. When sufficient colonies were visible, typically after 2-3 weeks, cells were washed twice in PBS before fixing in ice-cold 70% methanol for 30 minutes, stained by 0.2% Crystal violet for 2-3 hours. The following day cells were rinsed in PBS and air-dried. For senescence-associated β-galactosidase staining (SA-β-Gal), passage 21 MEFs were used and were fixed in 2% formaldehyde/0.2% glutaraldehyde in PBS for 10 minutes and stained for SA-β-Gal according to manufacturer's instructions (Cell Signaling) overnight at 37° C.

### Chromosome Spreading and Centrosome Staining Assays

For chromosome spreading assay, early-passage 3 phase Parkin WT and KO MEFs were treated with colcemid (10 μg/mL) for 2 hours to induce metaphase arrest. After shake-off, the mitosis cells were resuspended in 1 mL of 75 mM KCl for 30 minutes at 37° C., then fixed with 1 mL of Carnoy's fixative (3:1, methanol:glacial acetic acid) for 10 minutes, and then stained with 4′,6-diamidino-2-phenylindole (DAPI). The cells were collected by low-speed centrifugation (600 rpm) for 5 minutes, and then resuspended in an appropriate volume of fixative. The cell suspension was dropped onto glass slides in a humid condition chamber at 40-50° C. and spread cells were air-dried at 37° C. Metaphase spread chromosomes were imaged by Nikon fluorescent microscopy. For γ-tubulin staining assay to check centrosome numbers, Parkin WT and KO MEFs of passage 5 or 21 stage were cultured in 6 well plates on cover glass and stained by DAPI for chromosomes and γ-tubulin for centrosomes. Cells in metaphase were capture and counted by fluorescence microscopy.

### Immunohistochemistry

The tissue arrays include a lung tumor tissue microarray containing 400 pairs of human lung cancer and matched or unmatched normal adjacent tissue. All of step for IHC were prepared following standard protocol. Briefly, immunohistochemical cytokeratin staining was performed on formalin-fixed, paraffin embedded tissue using an indirect immunoperoxidase technique. Sections mounted on silanized slides were dewaxed in xylene, dehydrated in ethanol, boiled in 0.01 M citrate buffer (pH 6.0) for 20 minutes in a microwave oven and then incubated with 3% hydrogen peroxide for 5 minutes. After washing with PBS, the slides were incubated in 10% normal BSA for 5 minutes, followed by incubation for 45 minutes with rabbit polyclonal antibodies recognizing Parkin (ab15954, 1:200) and mouse monoclonal antibody recognizing anti-Plk1 (Invitrogen, 1:200). After washing, sections were incubated with labeled polymer (Bond Polymer Refine Detection) and diaminobenzidine. The sections were then counterstained with hematoxylin, dehydrated, cleared, and mounted.

### Doxycycline-Inducible Parkin Tet-On A549 Cell Lines

The pcDNA6/TR-Parkin was obtained from Dr. Nadja Patenge (Rothfuss et al., Hum. Mol. Genet., 18:3832-3850 (2009)). Subconfluent 1×10⁶ A549 cells were transfected with the pTet-On plasmid using Lipofetamine™ 2000 (Invitrogen, Carlsbad, Calif.). At 24 hours after transfection, the medium was removed, and cells were washed with 1×PBS at 37° C., and then supplemented with complete media containing 300 mg/mL of zeocin (Invitrogen) for selection of positive Parkin clones. Parkin expression was induced by the addition of 1-2 mg/mL doxycycline (Sigma) for 24 hours to the culture medium. The amount of Parkin protein was determined using immunobloting as described by the manufacturer (Lee et al., J. Cell Sci., 124:1911-1924 (2011)).

### Mouse Xenograft Tumor Model

For MEF xenograft experiments, equal numbers (1×10⁶ cells) of Parkin WT or KO MEF cells expressing luciferase mixed at a 1:1 dilution with matrigel (Collaborative Research) were implanted in the backs of athymic nude mice. Tumor growth was monitored using calipers and visualized with a bioluminescence-based IVIS system (Caliper LifeScience). For Parkin doxycycline-inducible xenograft experiments, 2×10⁶ A549 cells, stably transduced with a doxycycline-inducible Parkin construct (WT, S378A and S378D) or an empty virus, were re-suspended in matrigel and injected subcutaneously into athymic nude mice. Two days after injection, doxycycline was administered in drinking water. Tumour growth was measured using a vernier caliper at the indicated times after injection, and the tumor volume was calculated as length×width×height. For tumour xenograft experiments, nude mice were injected intradermally with 1×10⁶ Parkin WT or KO (with/without empty, Parkin WT, S378A and S378D) MEF cells. Nude mice bearing established Parkin WT or KO MEFs were treated i.v. for four cycles with either the vehicle control or BI 2536 at a dose of 20 mg/kg twice weekly on two. Tumor size was monitored by measuring mice two times a week. When tumors reached 2 cm in diameter, mice were killed.

### Statistical Analysis

Each assay was performed in triplicate and independently repeated at least three times. The results were presented as mean±standard error of mean (SEM). Statistical analyses were performed using GraphPad Prism software (version 4.02; GraphPad Software, San Diego, Calif.). One-way analysis of variance (ANOVA) followed by T-test was used to compare the results. A difference was considered significant if P<0.05. Statistical significance was defined as P<0.05 (*), P<0.01(**), and P<0.001(*** or ###).

### Parkin Regulates Mitosis

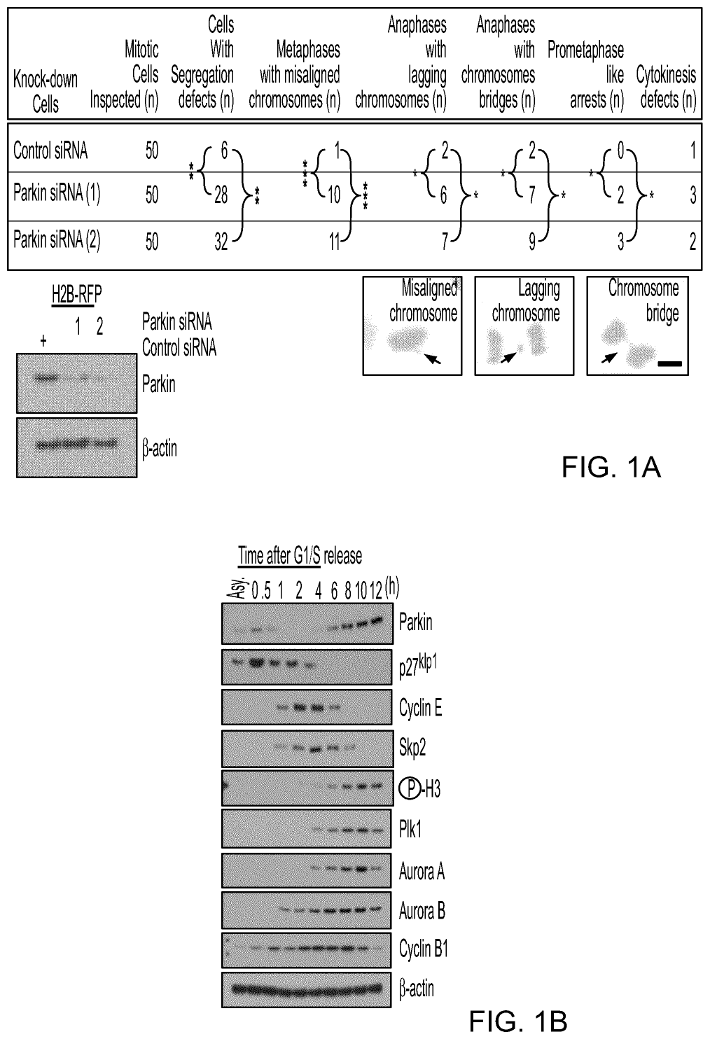

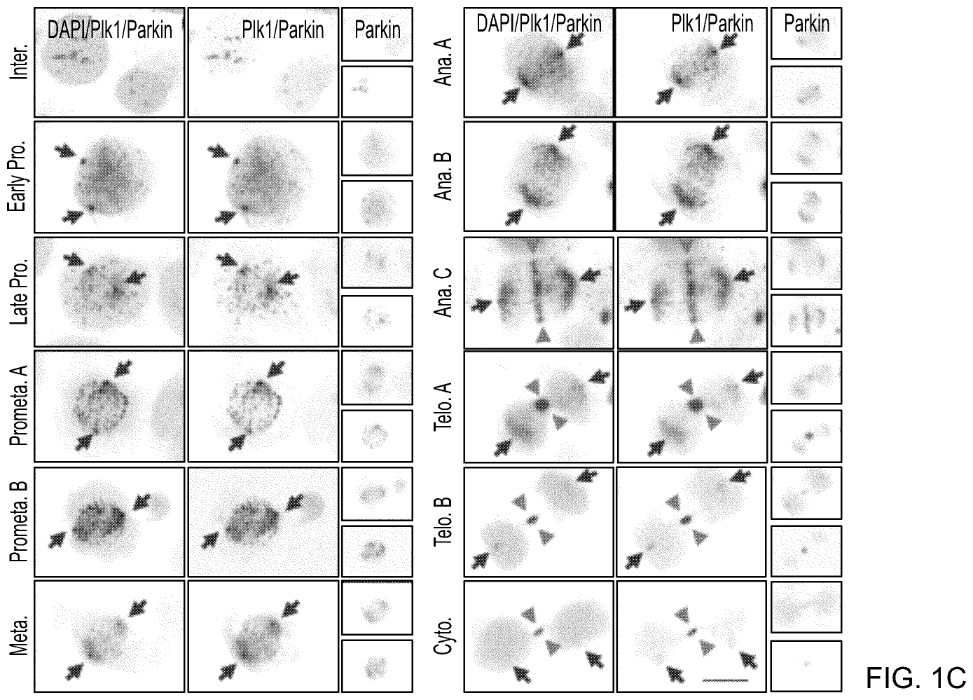

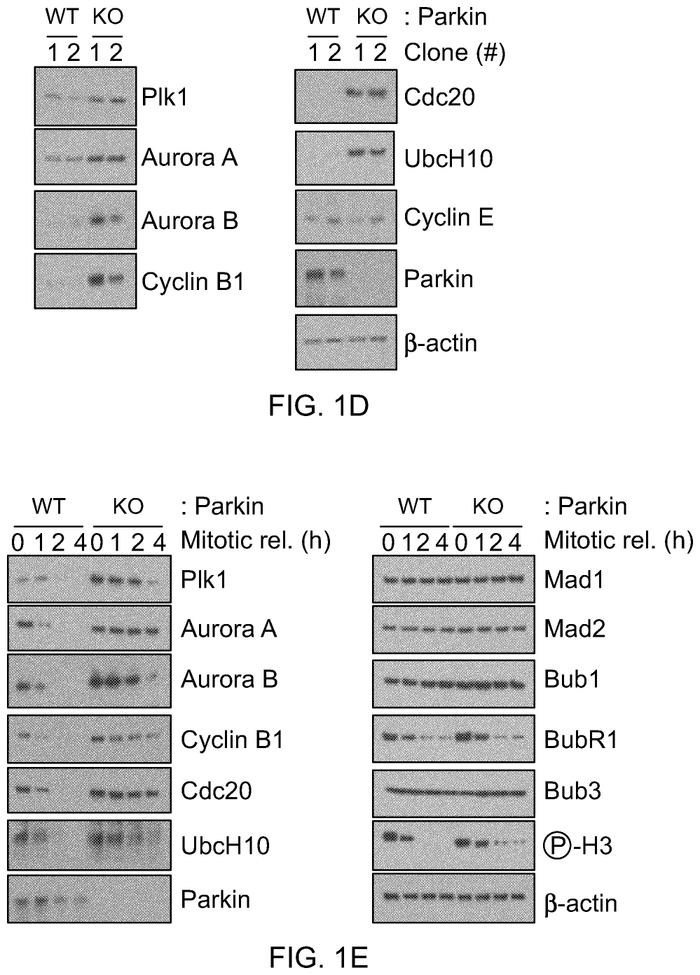

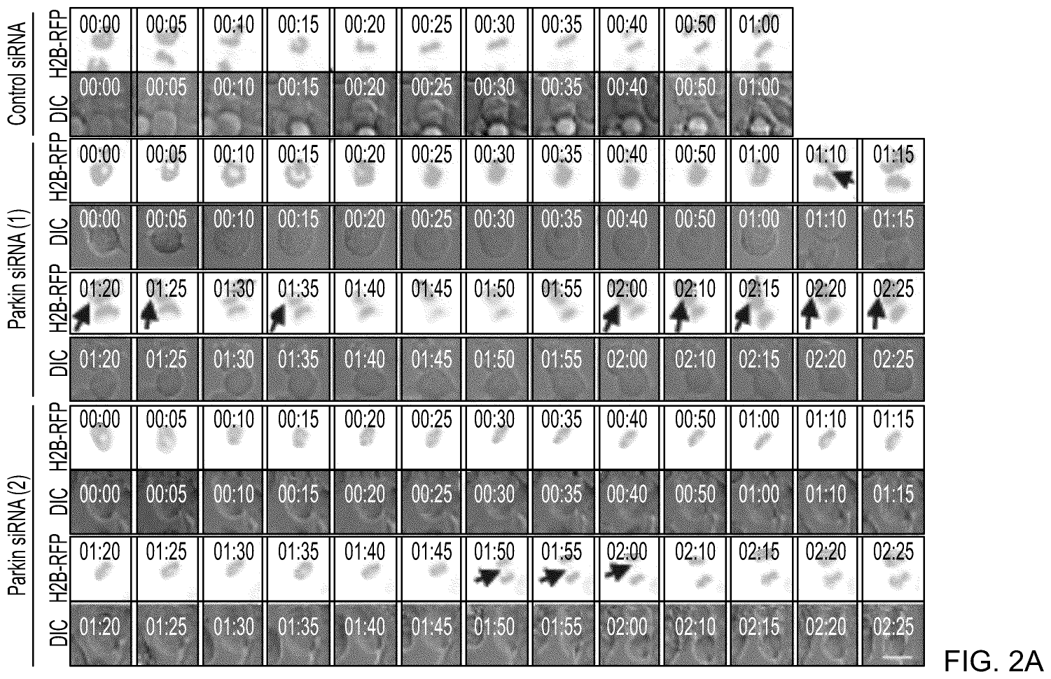

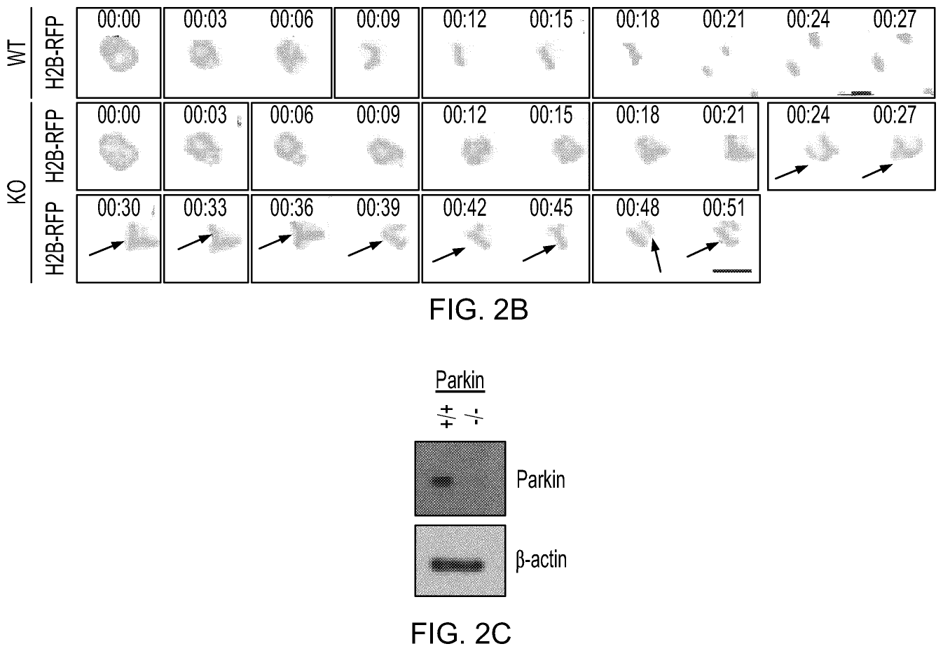

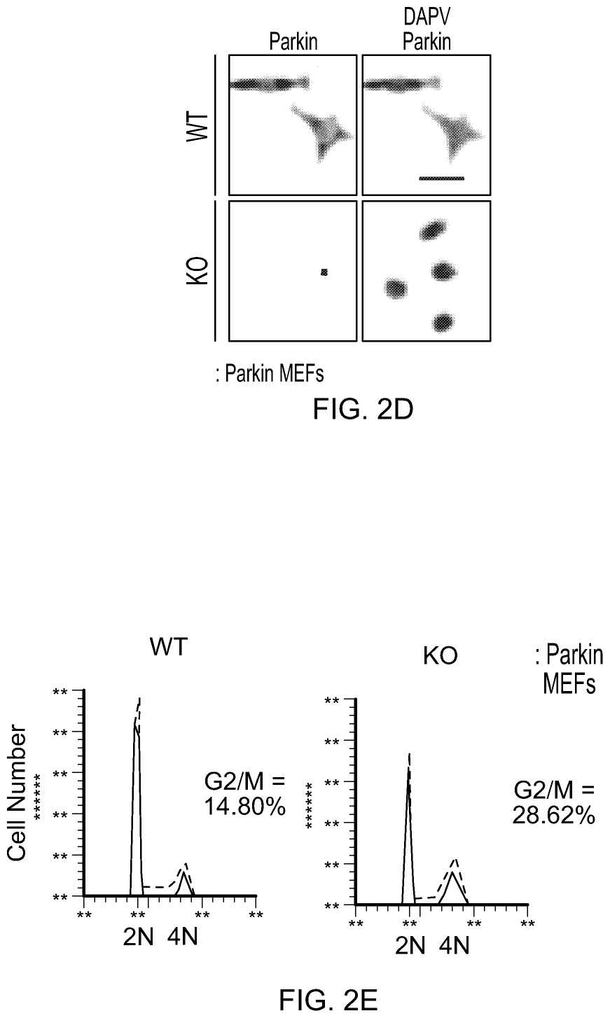

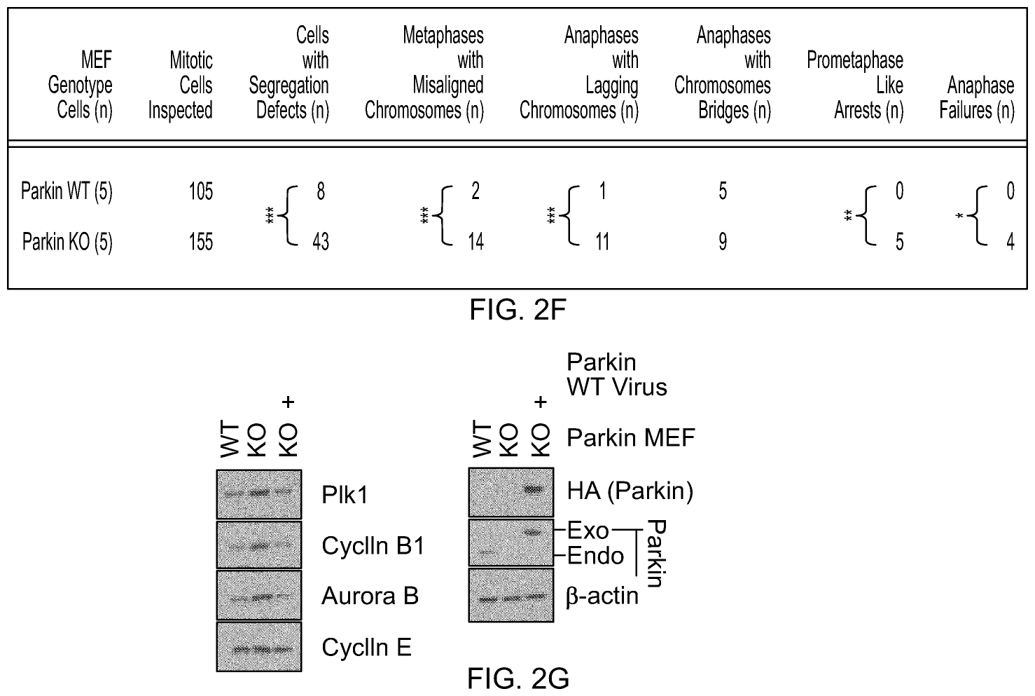

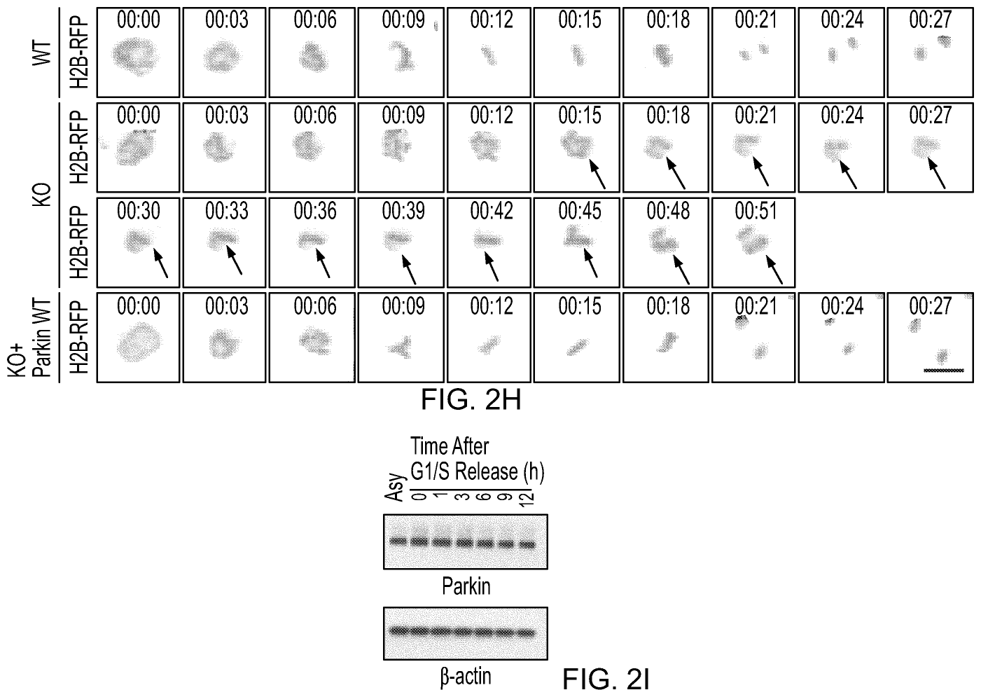

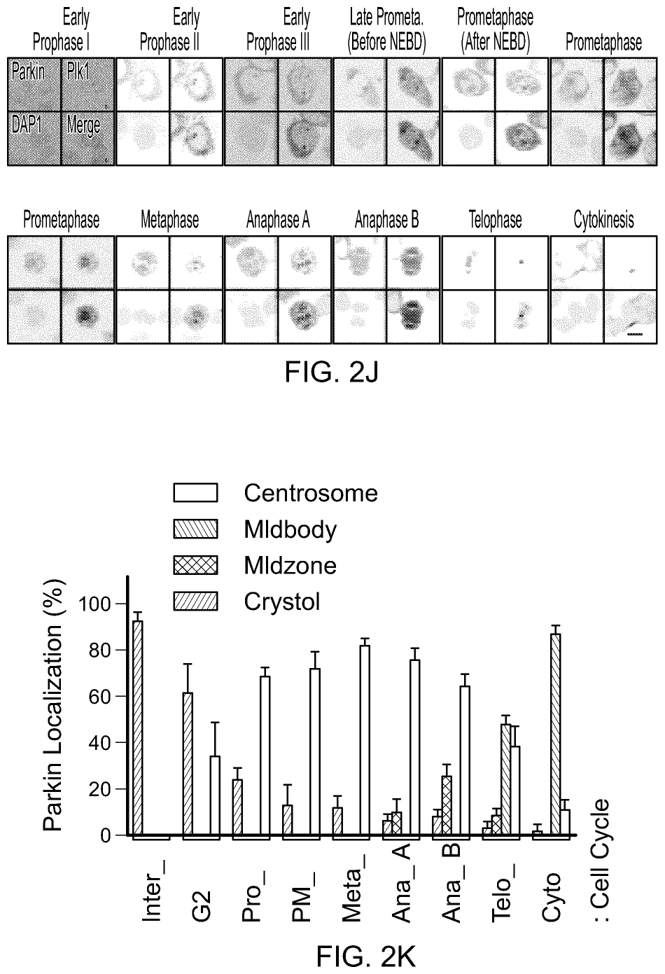

To understand the role of Parkin in mitosis, mitotic chromosome movement was monitored using time-lapse microscopy in Parkin-depleted U2OS cells (FIGS. 1A and 2A) and Parkin knockout (KO) mouse embryonic fibroblasts (MEFs; FIGS. 2B-2F). This analysis revealed a broad spectrum of mitotic defects including chromosome misalignment, chromosome lagging, chromosome bridge formation, prometaphase-like arrest, anaphase and cytokinesis failure (FIGS. 1A and 2F). In addition, progression from nuclear envelope breakdown (NEBD) to anaphase onset was significantly delayed in Parkin KO MEFs compared to wild type (WT) MEFs (FIGS. 2B and 2H), a defect that was reversed by exogenous expression of WT Parkin (FIGS. 2G and 2H). These results demonstrate that Parkin deficiency results in multiple mitotic defects.

Next, Parkin levels were examined at different stages of the cell cycle. Cells arrested at the G1/S boundary by double thymidine block (DTB) showed high Parkin levels. Upon release, Parkin levels decreased as cells progressed through S phase, and then peaked from G2 until early G1, without corresponding changes in mRNA levels (FIGS. 1B and 2I). Furthermore, Parkin was localized to centrosomes, midzone, and midbody in various cells types, including U2OS cells (FIGS. 1C, 2J, and 2K) and IMR-90 lung fibroblasts (PDL=33) (data not shown). These results suggest that Parkin might have a direct role in mitotic regulation.

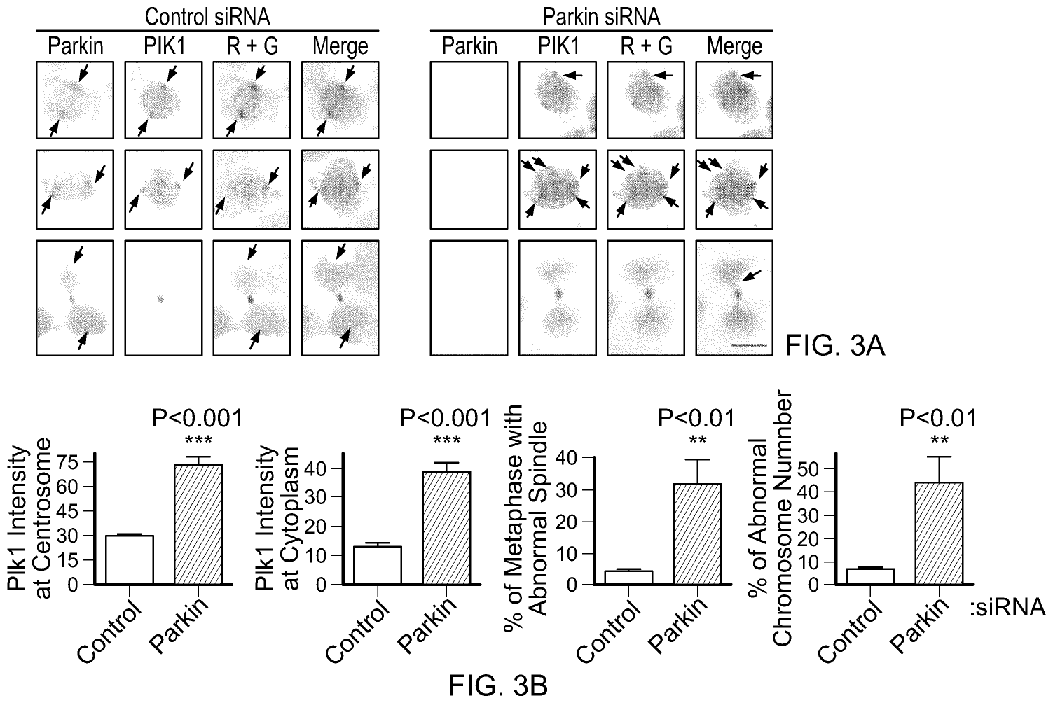

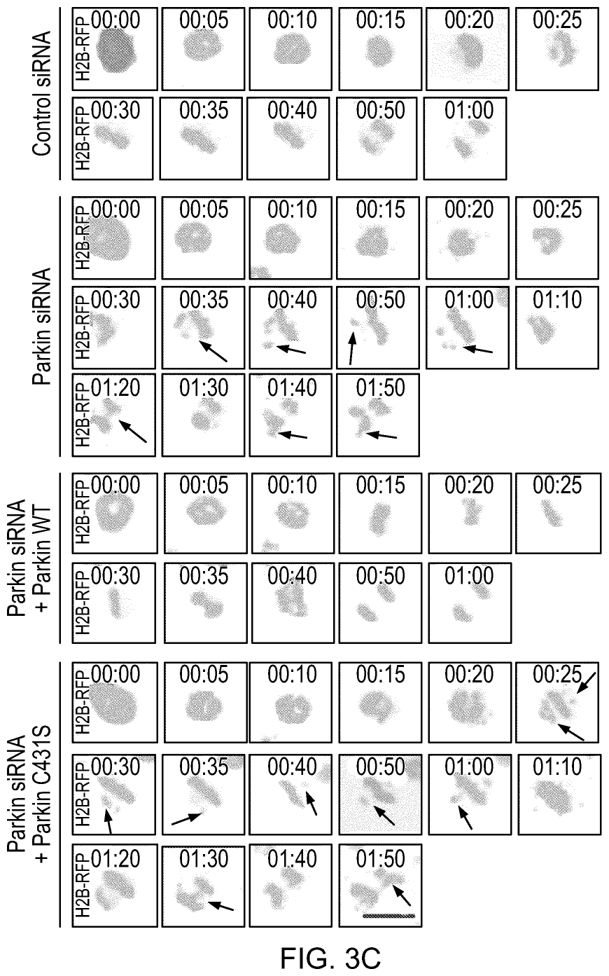

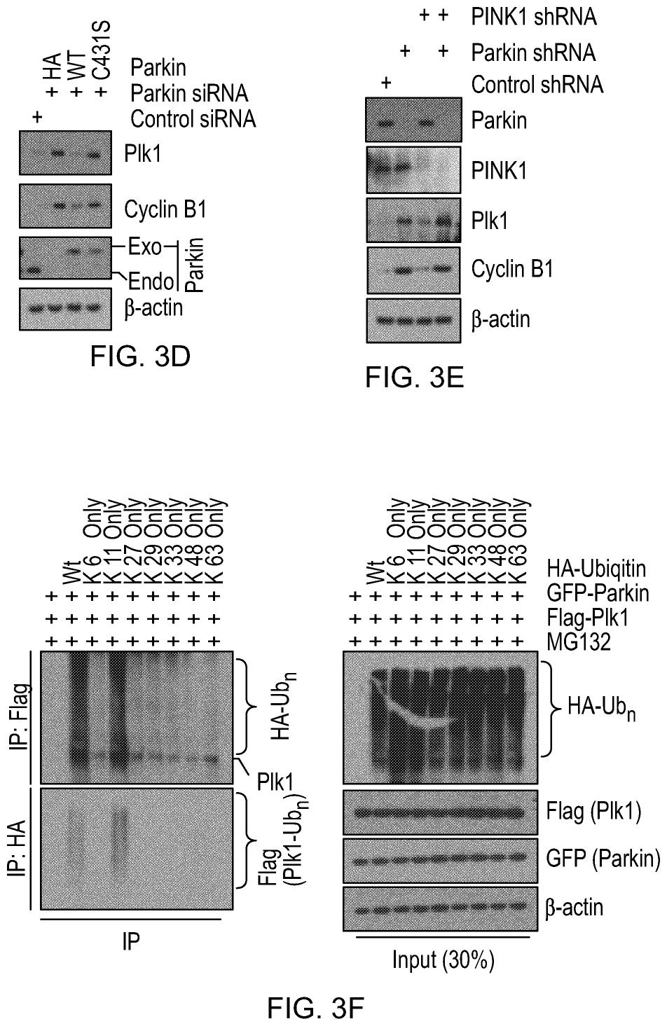

To examine how Parkin might regulate mitosis, the expression of key mitotic regulators was examined. Immunoblot analysis of asynchronous or mitotic lysates from Parkin WT and KO MEFs showed increased levels of Plk1, Aurora A, Aurora B, Cyclin B1, Cdc20, and UbcH10 (FIGS. 1D and E). Other key mitotic regulators, such as Mad1, Mad2, Bub1, BubR1 and Bub3 were not affected. Cyclin E, whose upregulation has been linked to genomic instability in Parkin-deficient cells (Veeriah et al., Nat. Genet., 42:77-82 (2010)), was also present at normal levels. Furthermore, Parkin-depleted cells showed aberrant localization and expression of Plk1, Cyclin B1, and Aurora B as examined by immunofluorescence (IF) and immunoblot (FIGS. 3A and 3B; data not shown), respectively. Mitotic defects and up-regulation of Plk1 and Cyclin B1 in Parkin-depleted cells were reversed by expressing WT Parkin but not C431S, which abolishes Parkin's E3 ligase activity (FIGS. 3C and 3D) (Iguchi et al., J. Biol. Chem., 288:22019-22032 (2013); and Riley et al., Nat. Commun., 4:1982 (2013)). These results suggested that Parkin regulates mitosis by controlling the levels of particular mitotic regulators through its E3 ligase activity. PINK′ knockdown did not affect Plk1 and Cyclin B1 levels, suggesting that Parkin's role in mitotic regulation is PINK1-independent (FIG. 3E), and thus distinct from Parkin's established role in mitophagy.

### Parkin Mediated Ubiquitination is a Mitotic Regulator

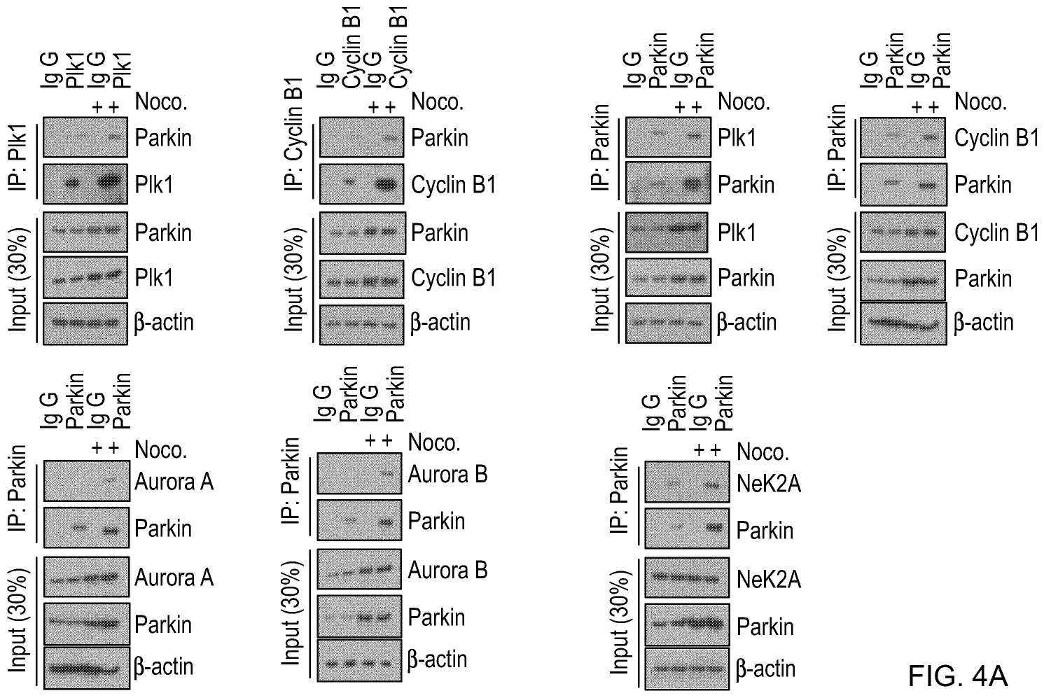

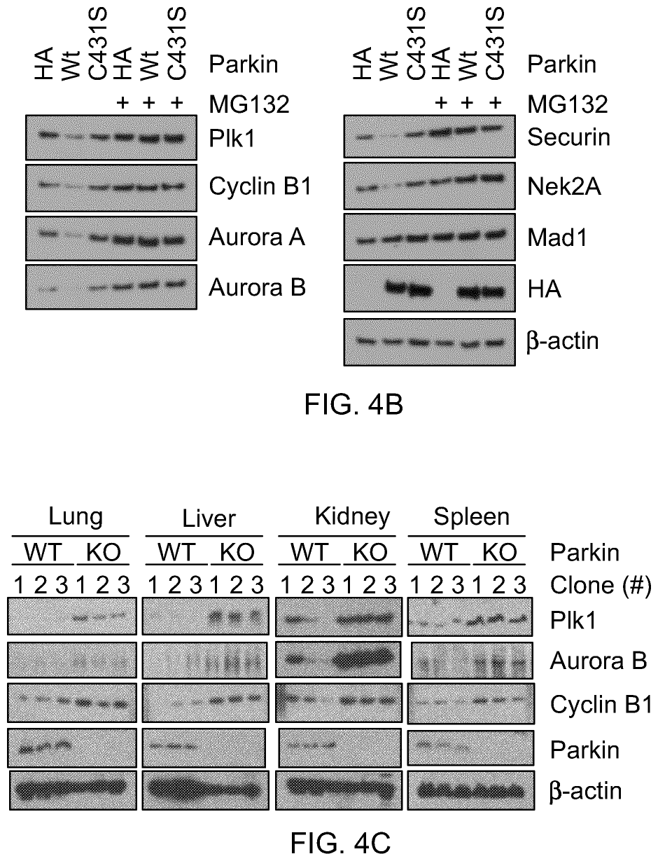

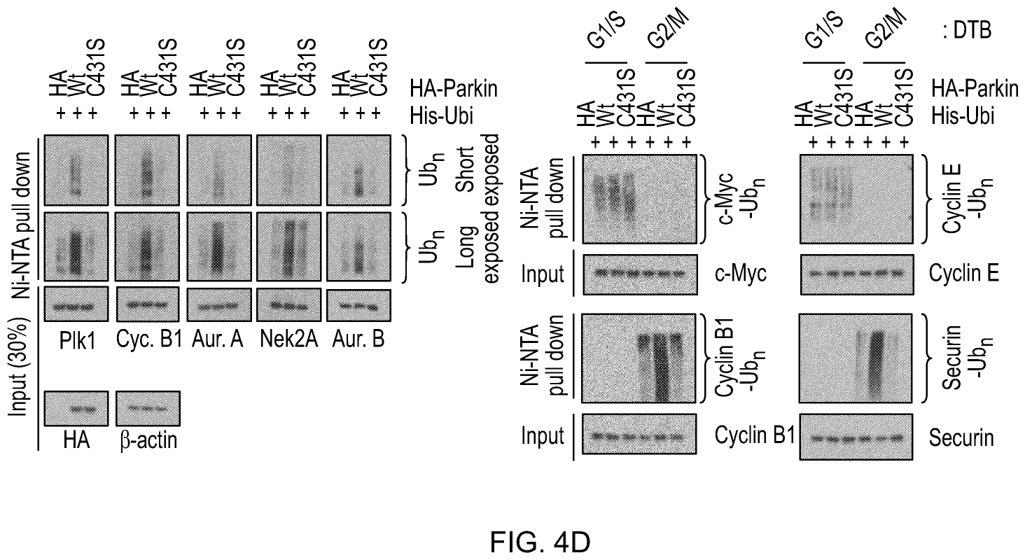

It was hypothesized that Parkin directly regulates the levels of mitotic regulators, such as Plk1 and Aurora B, through its E3 ligase activity (Shimura et al., Nat. Genet., 25:302-305 (2000)). Endogenous Parkin interacts with Plk1, Cyclin B1, Aurora A, Aurora B, and Nek2A (FIG. 4A). Furthermore, overexpression of Parkin WT, but not C431S mutant, markedly decreased levels of these mitotic regulators, which could be prevented by MG132 pre-treatment (FIG. 4B), supporting the idea that Parkin regulates the abundance of these mitotic regulators through the proteasome pathway. Immunoblot analysis of tissue lysates from Parkin WT and KO mice revealed that Plk1, Aurora B, and Cyclin B1 protein levels are elevated in tissues lacking Parkin (FIG. 4C). Importantly, overexpression of Parkin in cells increased the polyubiquitination of Plk1, Aurora B, Cyclin B1, Aurora A, Securin, Aurora B, and Nek2A, but not c-Myc and Cyclin E, whose expression was not regulated by Parkin (FIG. 4D). Furthermore, the C431S mutation abolished Parkin's E3 ligase activity toward its substrates. Early studies suggest that Parkin mediates K48- or K63-linked polyubiquitylation in brain (Moore et al., J. Neurochem., 105:1806-1819 (2008); Olzmann et al., J. Cell. Biol., 178:1025-1038 (2007); Youle and Narendra, Nat. Rev. Mol. Cell. Biol., 12:9-14 (2011)). Interestingly, Parkin mostly mediated K11-linked polyubiquitin-chains in Plk1 ubiquitination (FIG. 3F). Collectively, these results indicate that Parkin regulates the levels of a subset of mitotic proteins through the ubiquitin-proteasome pathway.

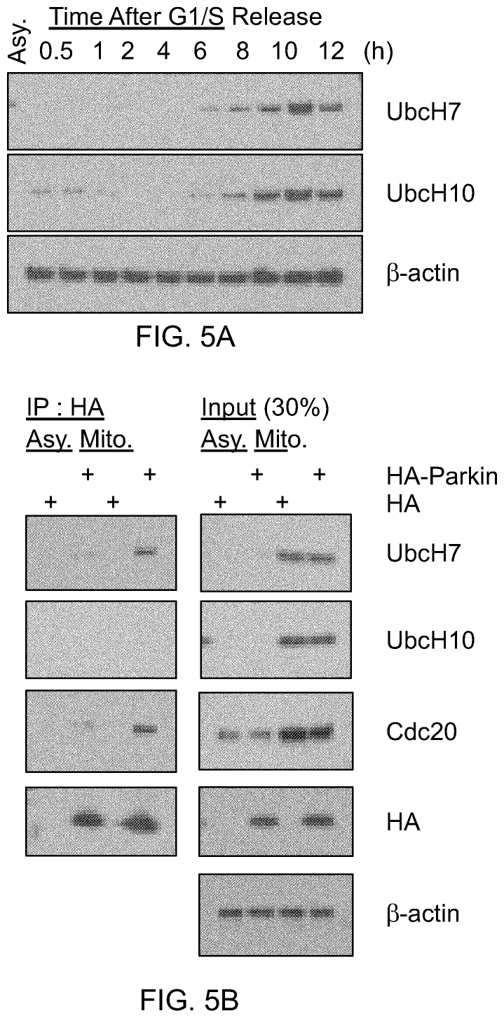

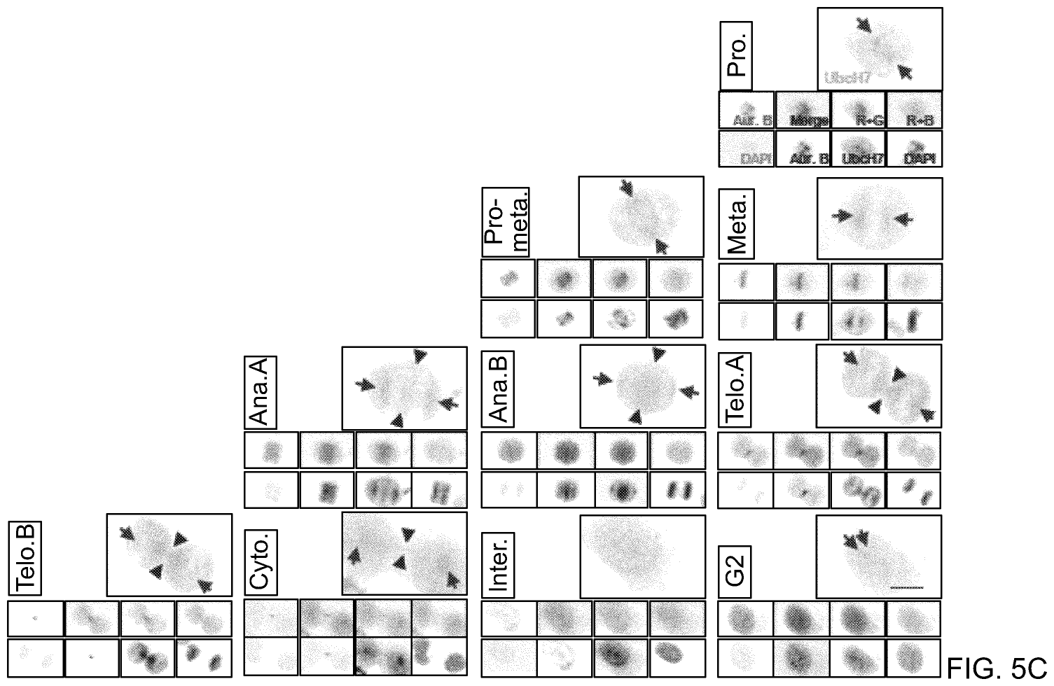

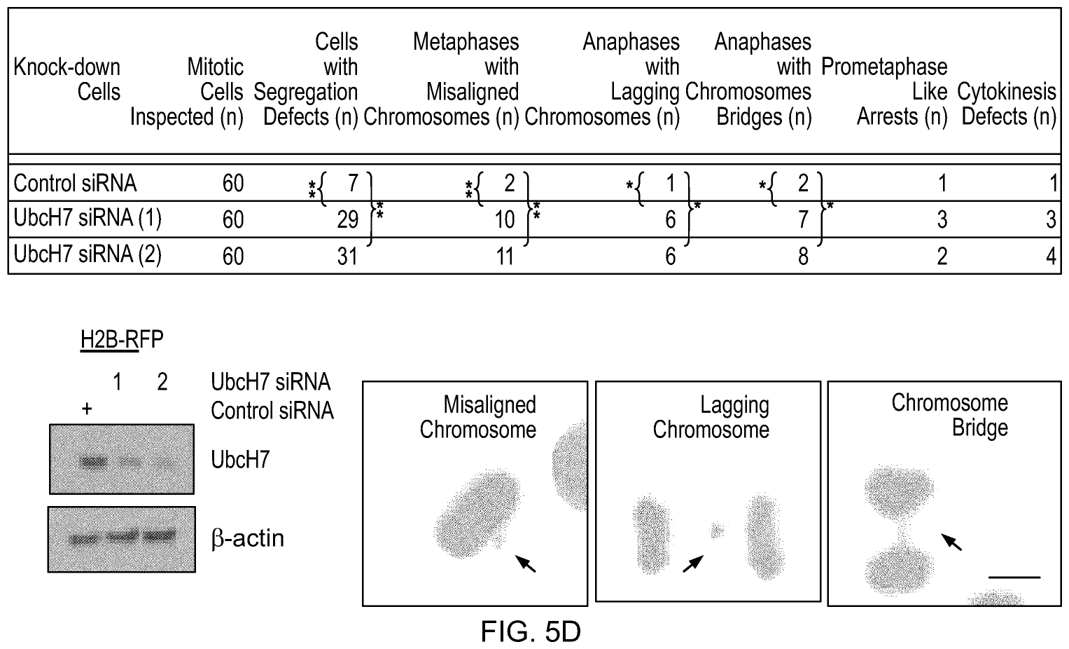

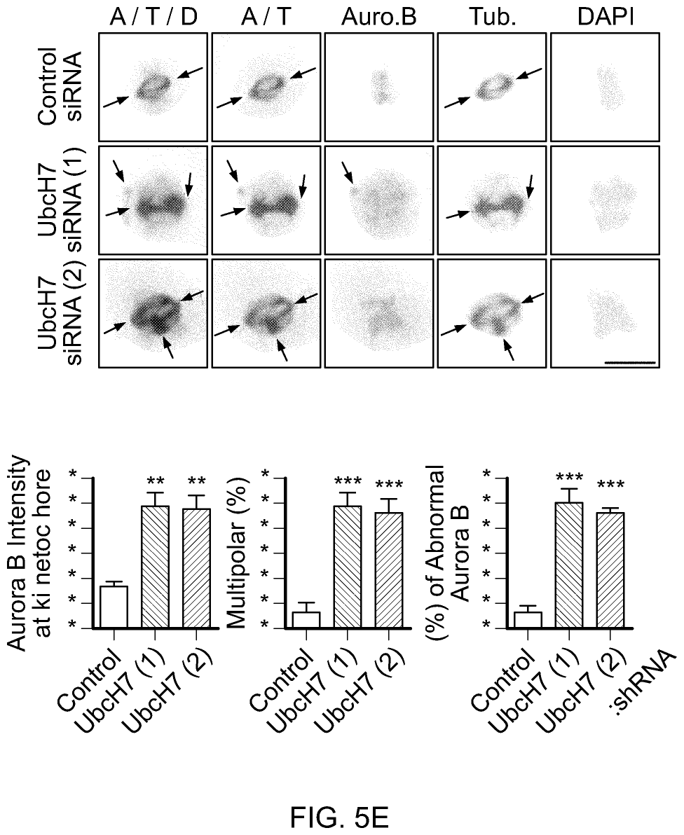

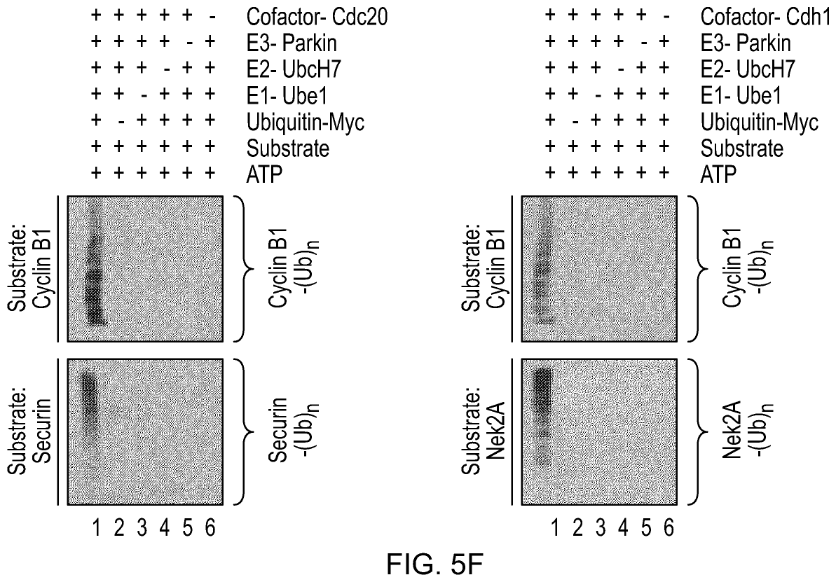

In experiments designed to identify the E2 ubiquitin ligase for Parkin, an interaction was not observed between Parkin and UbcH10, the E2 for APC/C in mitosis (data not shown) (Castro et al., Oncogene, 24:314-325 (2005); and Peters, Nat. Rev. Mol. Cell. Biol., 7:644-656 (2006)). Instead, UbcH7 (also called Ube2L3), the E2 for Parkin in cellular processes other than mitosis (Shimura et al., Nat. Genet., 25:302-305 (2000); and Wenzel et al., Nature, 474:105-108 (2011)), was significantly elevated and interacted with Parkin in mitosis (FIGS. 5A and 5B) and accumulated at various mitotic structures, including centrosomes, midzone, and midbody, just like Parkin (FIG. 5C). Importantly, UbcH7 depletion caused mitotic defects similar to Parkin depletion (FIGS. 5D and 5E), further supporting the idea that UbcH7 acts as an E2 ubiquitin ligase for Parkin in mitosis.

### Parkin-Cdc20/Cdh1 Acts as a Mitotic-Regulating Complex

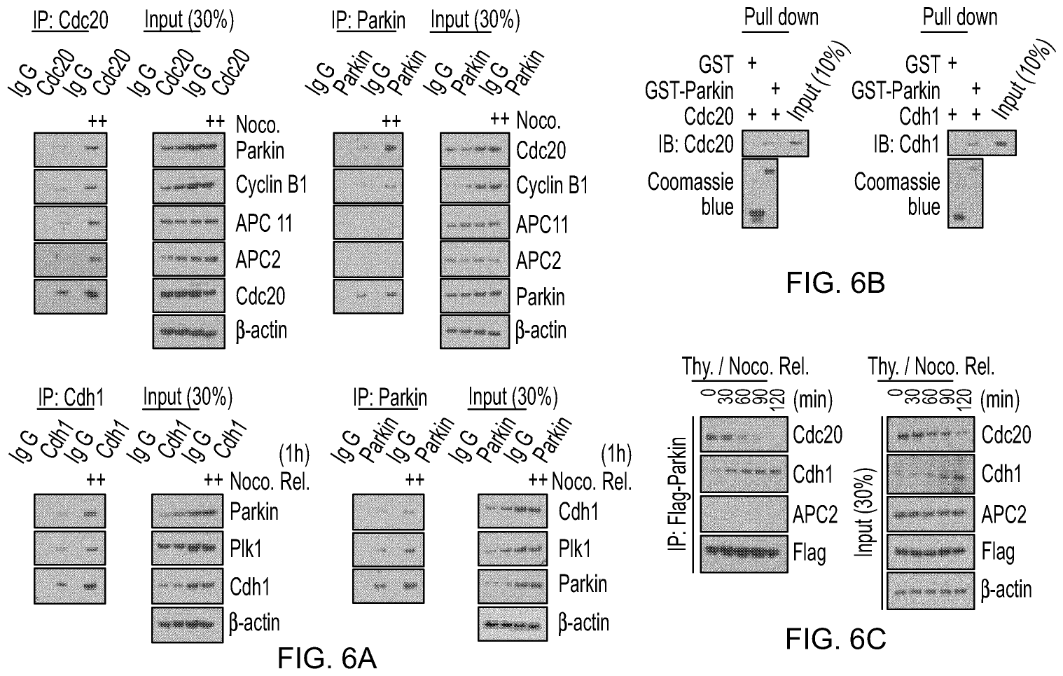

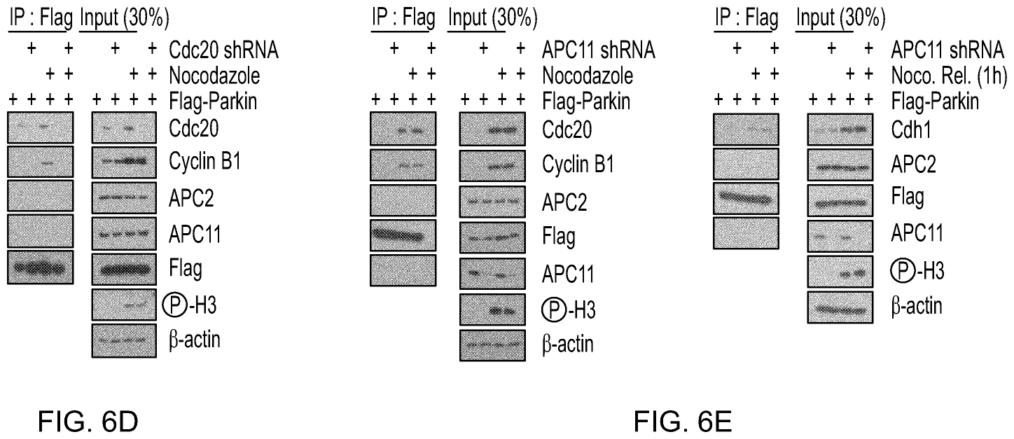

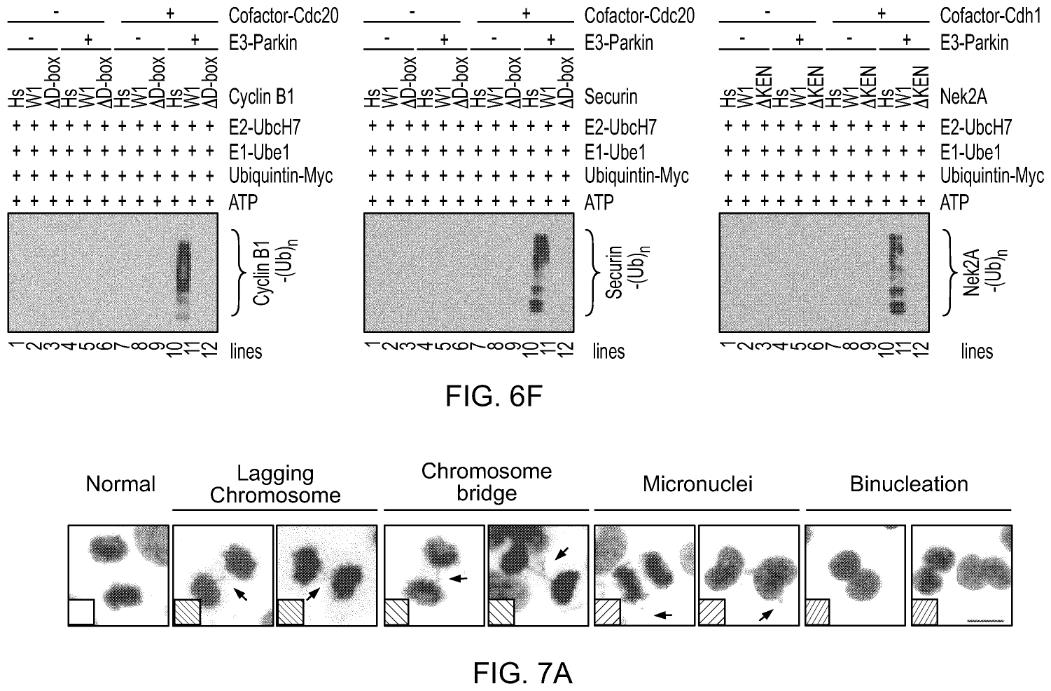

Parkin regulates mitotic factors, which are also regulated by APC/C, raising the possibility that Parkin interacts with APC/C or its subunits. The interaction between Parkin and the APC/C subunits was examined (FIG. 6A). Endogenous Parkin co-immunoprecipitated with Cdc20 and Cdh1 from mitotic cell lysates, but not with APC/C components APC11 and APC2. Furthermore, recombinant Parkin interacted with Cdc20 and Cdh1 under cell-free conditions, suggesting that Parkin directly interacts with Cdc20/Cdh1 (FIG. 6B). Use of synchronized cell lysates indicated that Parkin first interacts with Cdc20 and then switches to Cdh1 after cells exit mitosis (FIG. 6C). Taken together, these results suggest that Parkin forms a complex with Cdc20 or Cdh1 that does not include the APC/C.

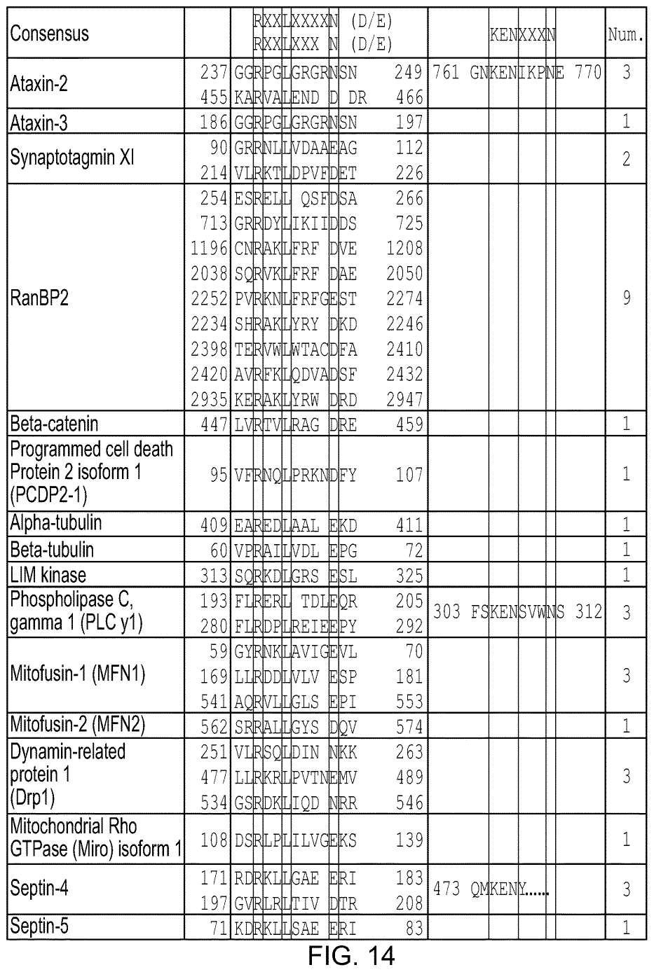

Cdc20 and Cdh1 act as substrate-recognition subunits of APC/C (Castro et al., Oncogene, 24:314-325 (2005); and Peters, Nat. Rev. Mol. Cell. Biol., 7:644-656 (2006)). Parkin might also target specific mitotic substrates through Cdc20 and Cdh1. Knockdown of Cdc20 or Cdh1 resulted in decreased binding of Parkin to various mitotic substrates, including Cyclin B1 and Aurora B (FIG. 6D and data not shown). In contrast, knockdown of APC11 did not affect these interactions and Parkin's interaction with Cdc20/Cdh1 (FIG. 6E). Moreover, Cdc20- and Cdh1-specific degron sequences (D-box and KEN box motifs) (Castro et al., Oncogene, 24:314-325 (2005); and Nakayama and Nakayama, Nat. Rev. Cancer, 6:369-381 (2006)) were found in a series of established Parkin substrates, including Ataxin 2 and 3, Synaptotagmin XI, RanBP2, β-catenin, PCDP2-1, α and β tubulin, LIM kinase, PLC-γ1, MFN1 and 2, Mitochondrial Rho GTPase isoform 1, Septin 4 and 5, and Drp1 (FIG. 14) (Walden and Martinez-Tones, Cell. Mol. Life Sci., 69:3053-3067 (2012)), the latter of which was shown to require Cdh1 for ubiquitination (Horn et al., Mol. Biol. Cell., 22:1207-1216 (2011); and Wang et al., J. Biol. Chem., 286:11649-11658 (2011)). To further confirm the role of Cdc20 and Cdh1 in Parkin-mediated ubiquitination, in vitro ubiquitination assays were performed. Parkin induced ubiquitination of Cyclin B1, Securin and Nek2A; however, their ubiquitination were abolished in the absent of Cdc20/Cdh1, Ube1 (E1), UbcH7 (E2) or Parkin (FIGS. 5F and 6F). Furthermore, the D-box/KEN-box mutants of these substrates were not polyubiquitinated by Parkin. These findings further strengthen the notion that Parkin-Cdc20 and -Cdh1 complexes act independently of APC/C-Cdc20 and -Cdh1 in regulating the abundance of key mitotic regulators.

### UbcH7-Parkin-Cdc20 and -Cdh1 Complexes Regulate Mitosis Independently of UbcH10-APC/C-Cdc20 and -Cdh1 Complexes

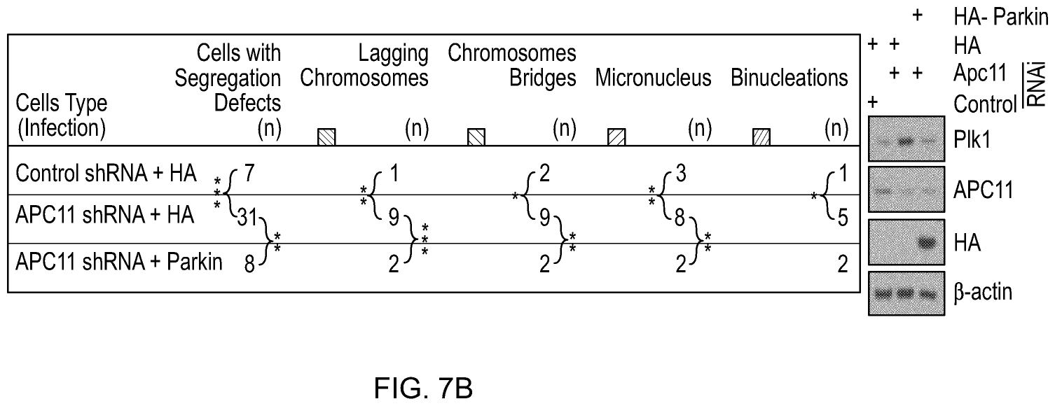

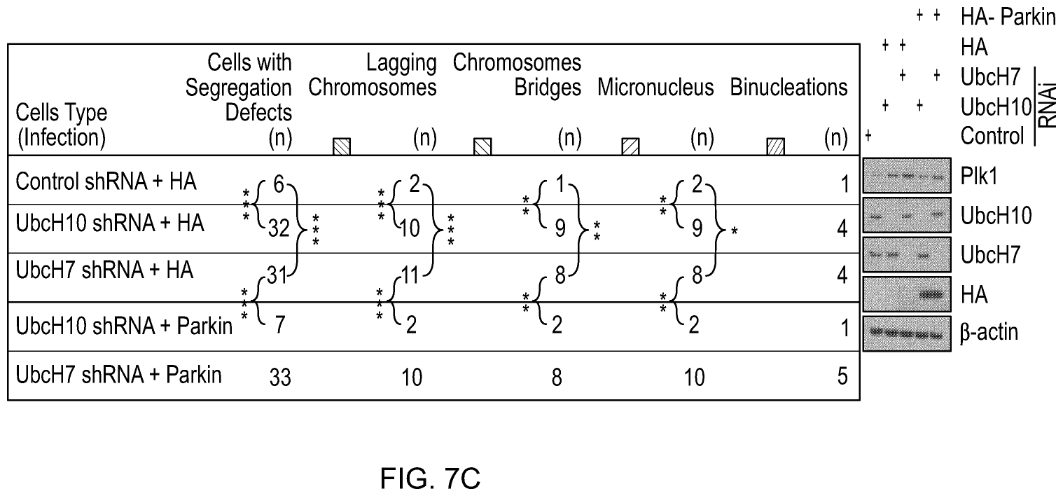

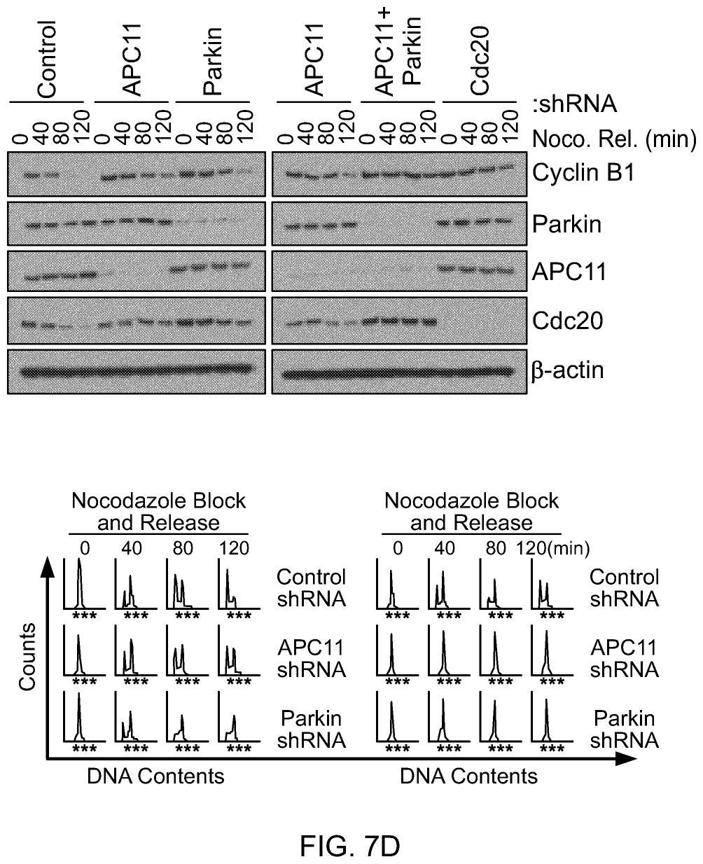

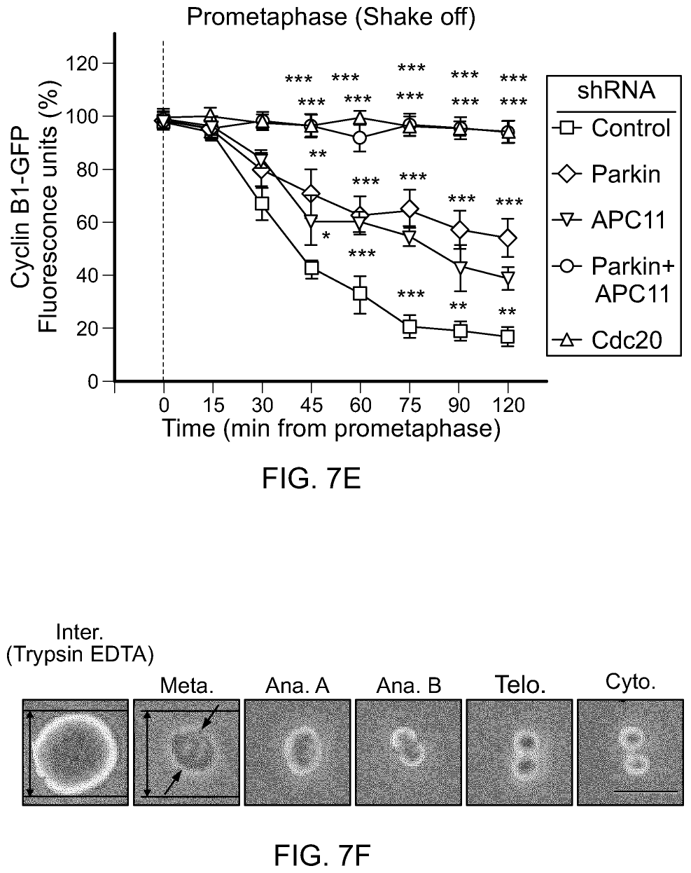

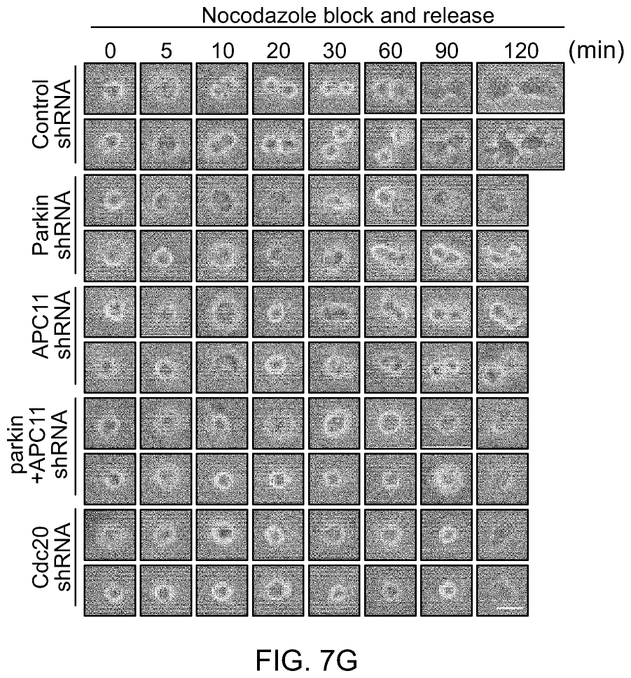

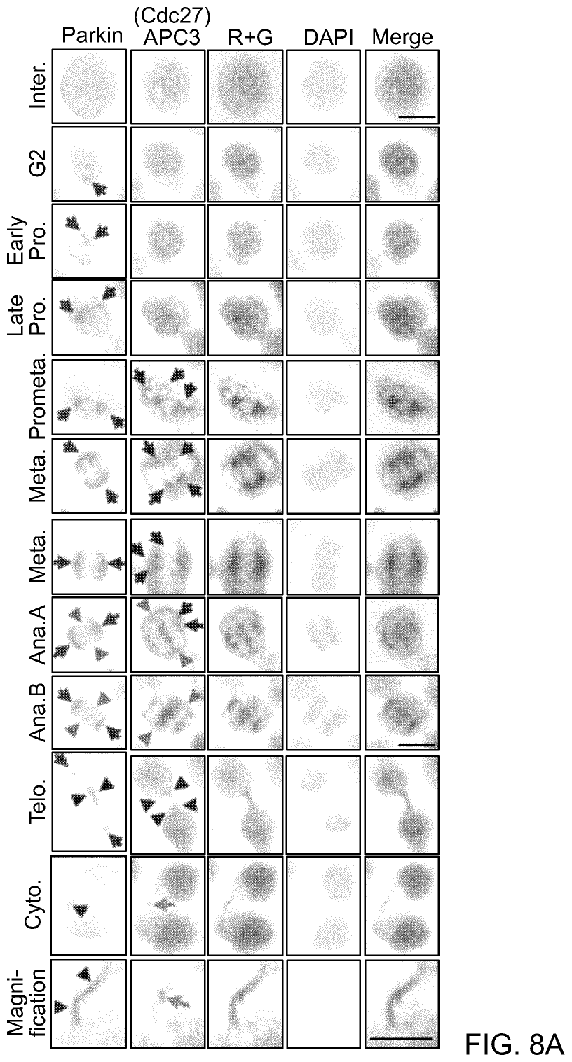

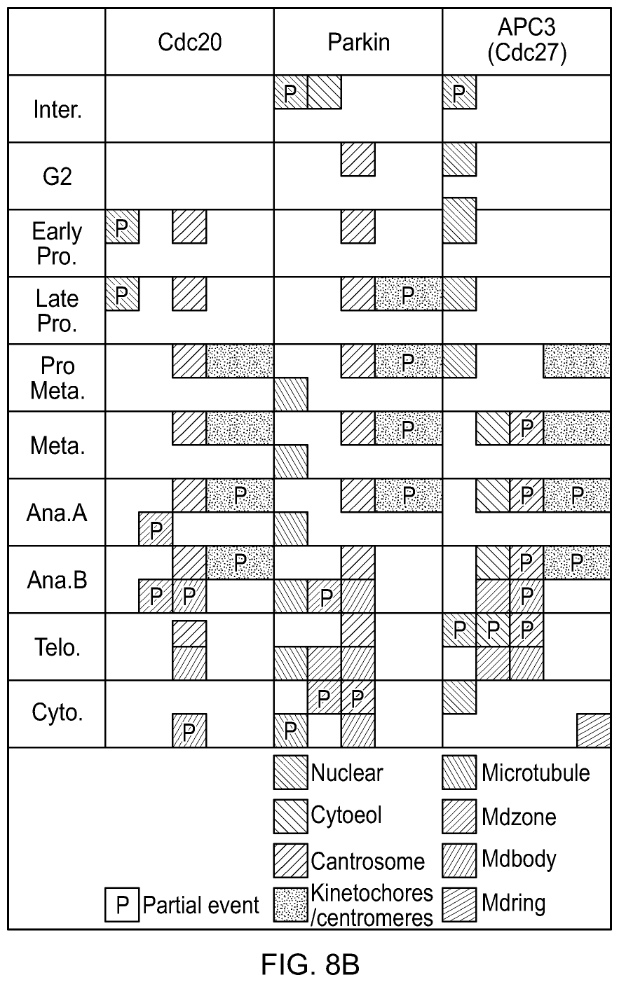

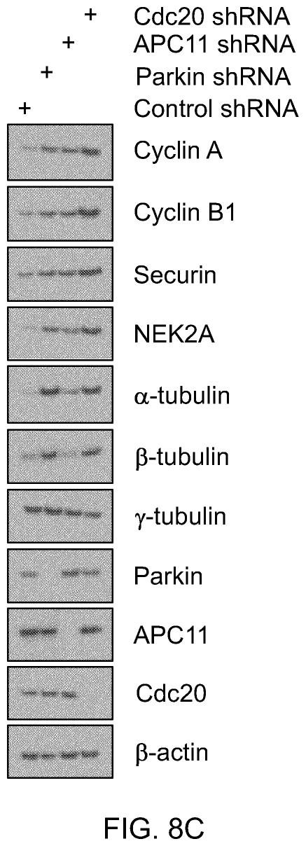

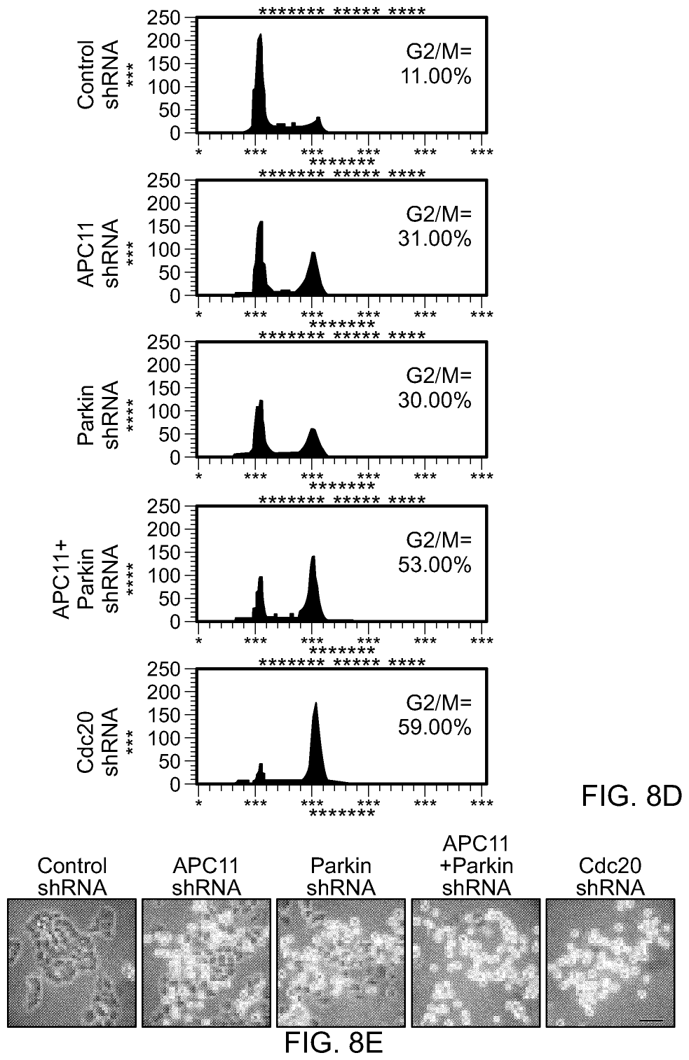

The functional interaction between Parkin and APC/C were examined. Inactivation of APC/C by APC11 knockdown resulted in chromosome missegregation defects and upregulation of Plk1 (FIGS. 7A and 7B). Ectopic expression of Parkin in APC11-deficient cells reversed these mitotic abnormalities (FIGS. 7A and 7B). In addition, Parkin overexpression restored Plk1 levels and rescued mitotic errors induced by UbcH10 (APC/C E2) knockdown, but had no effect on UbcH7 (Parkin's E2)-induced mitotic defects (FIG. 7C). These studies suggest that the UbcH7-Parkin-Cdc20 and -Cdh1 complexes regulate mitosis independently of UbcH10-APC/C-Cdc20 and -Cdh1 complexes. Although Parkin and APC/C show many similarities in mitosis, there are some differences in their localization. As shown in FIGS. 8A and 8B, Parkin is localized in the centrosome or midbody like Cdc20, while APC3 is localized in the kinetochores, or the midring in mitosis. Furthermore, UbcH7-Parkin-Cdc20 has target proteins such as α and β tubulin that are not regulated by APC/C (FIG. 8C).

Since Parkin and APC/C share the same coactivator Cdc20, one prediction is that mitotic defects caused by depletion of APC/C or Parkin alone would be less severe than those caused by depletion of Cdc20 (Huang et al., Cancer Cell, 16:347-358 (2009)). To test this idea, whether Parkin affects Cdc20-mediated degradation of Cyclin B1 at the metaphase-to-anaphase transition was studied. Depleting APC11 or Parkin alone delayed Cyclin B1 degradation and mitotic exit, but did not recapitulate Cdc20 depletion (FIGS. 7D, 7E, 7G, and 8C-8E). However, co-depletion of APC11 and Parkin phenocopied Cdc20 depletion (FIGS. 7D, 7E, 7F, 7G, 8D, and 8E).

### Parkin is Phosphorylated and Activated by Plk1 Upon Mitotic Entry

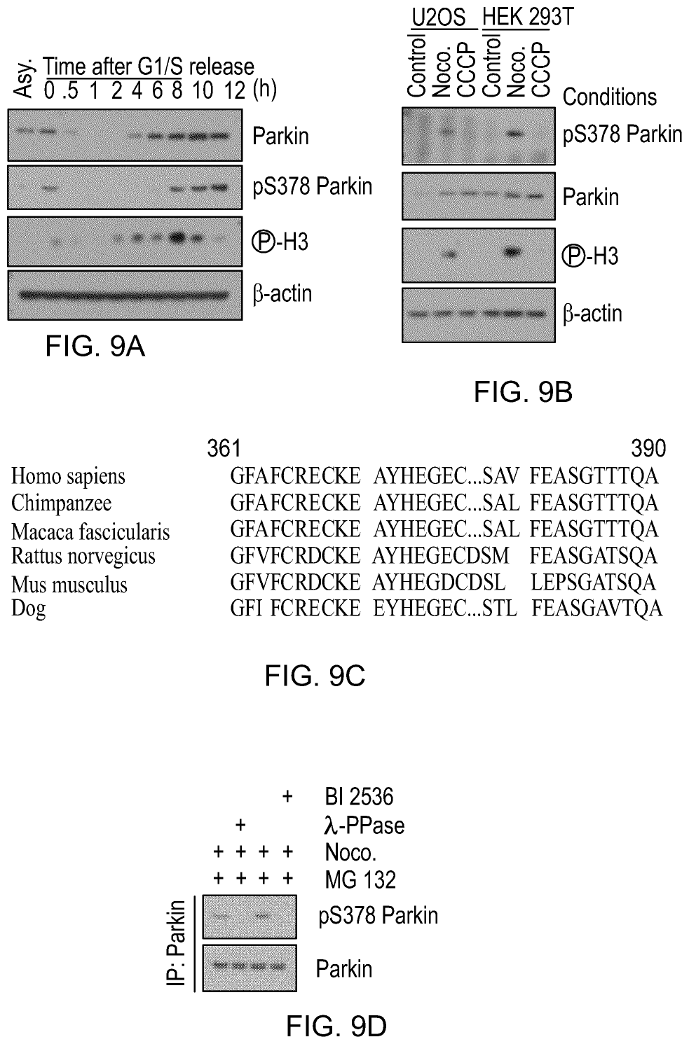

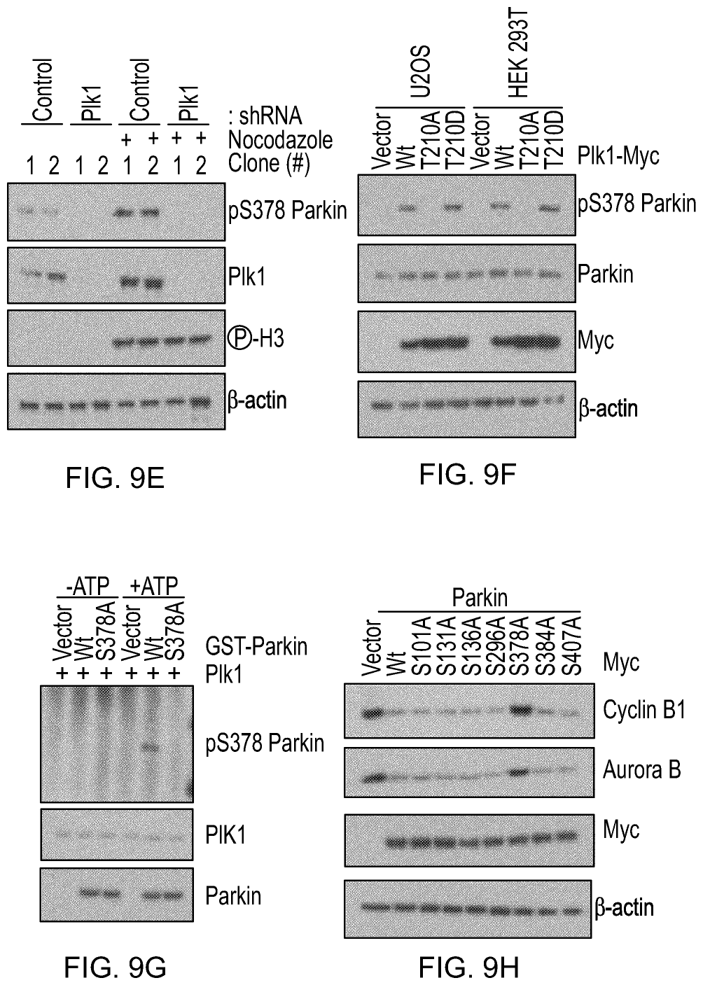

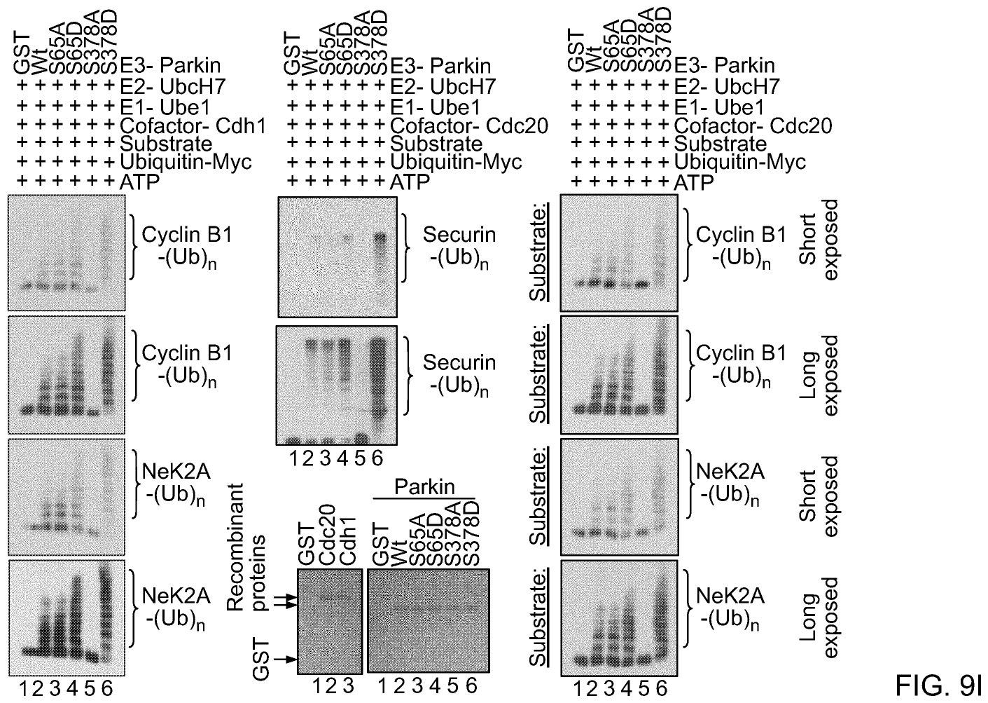

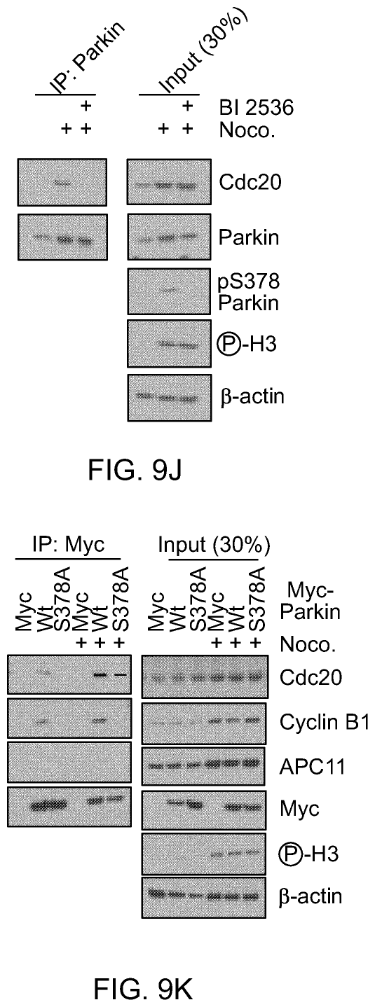

The following was performed to identify mitosis-specific regulation of Parkin. Phosphorylation is a common posttranslational modification and has been shown to involve protein stability and activity. Parkin was scanned using GPS2.12, a tool for prediction of kinase-specific phosphorylation sites (Xue et al., Mol. Cell. Proteomics, 7:1598-1608 (2008)), which identified Ser 378 as a potential phosphorylation site by Plk1. Parkin was phosphorylated at Ser 378 in mitosis (FIGS. 9A and 9B). Treatment of carbonyl cyanide m-chlorophenylhydrazone (CCCP), a mitochondrial-uncoupling reagent that activates Parkin during mitophagy (Iguchi et al., J. Biol. Chem., 288:22019-22032 (2013); and Riley et al., Nat. Commun., 4:1982 (2013)), did not affect Ser 378 phosphorylation (FIG. 9B). Ser 378 is predicted to be a Plk1 phosphorylation site (“gps.biocuckoo.org/”), and its surrounding residues fit with a consensus Plk1 phosphorylation site (D/ExS/TΦ, Φ: hydrophobic residues). In addition, Ser 378 is highly conserved among vertebrates (FIG. 9C), suggesting that the phosphorylation of this site may have an evolutionarily conserved role in regulating Parkin activity. To test whether Plk1 regulates Parkin S378 phosphorylation, cells were treated with BI 2536, a Plk1 inhibitor, or cells were infected with Plk1 shRNA. Plk1 inhibition or deficiency blocked Parkin phosphorylation at Ser378 (FIGS. 9D and 9E). Conversely, overexpression of Plk1 or constitutively active Plk1 (T210D) (van de Weerdt et al., Mol. Cell. Biol., 25:2031-2044 (2005)), but not inactive Plk1 (T210A), increased Parkin phosphorylation (FIG. 9F). Furthermore, Plk1 was able to phosphorylate recombinant WT Parkin but not S378A (FIG. 9G). Interestingly, Ser 378 localized within the IBR domain. In previous studies, it was established that the IBR domain assists the recruitment of proteins involved in the ubiquitination pathway (Chung et al., Nat. Med., 7:1144-1150 (2001); and Zhang et al., Proc. Natl. Acad. Sci. USA, 97:13354-13359 (2000)). Structurally, the IBR domain helps a close arrangement of the RING1 and RING2 domains, which facilitates protein interactions and subsequent ubiquitination (Beasley et al., Proc. Natl. Acad. Sci. USA, 104:3095-3100 (2007)). In addition, the region is involved in maintaining conformational flexibility, and it can affect Parkin's activity and stability (Trempe et al., Science, 340:1451-1455 (2013)).

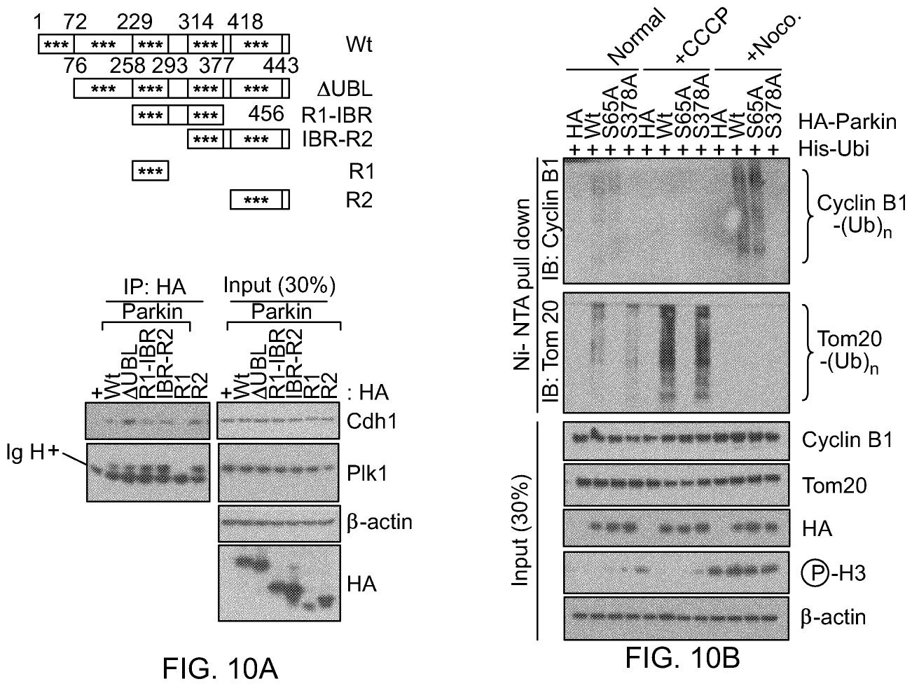

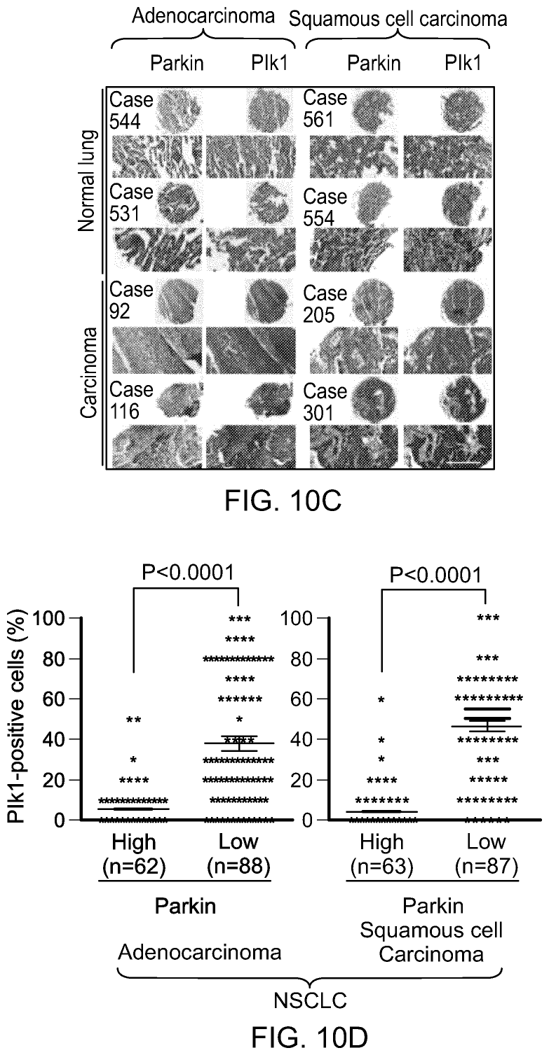

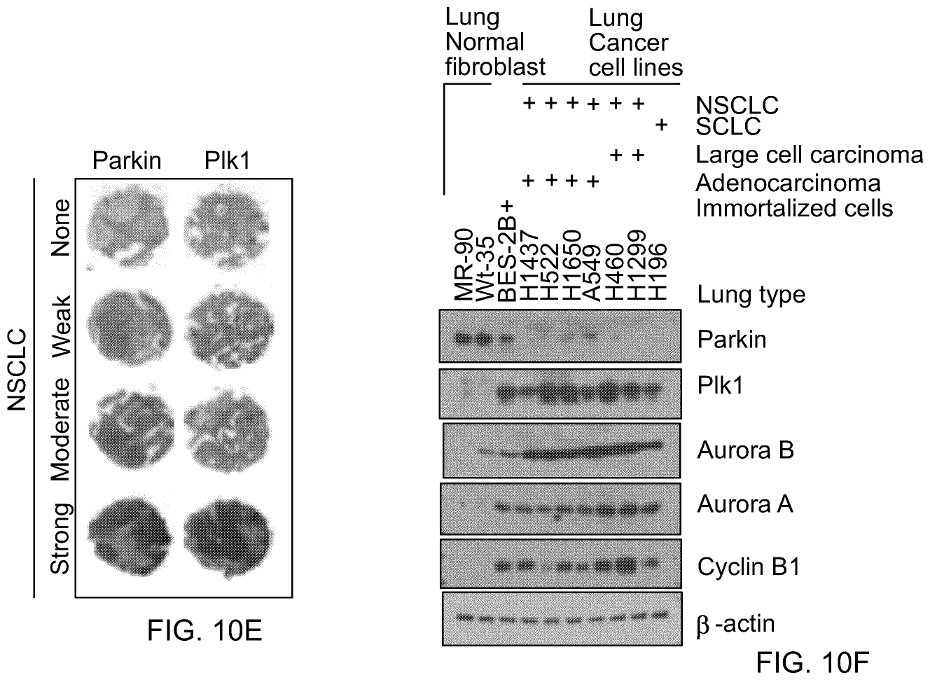

The IBR domain also was involved in Parkin's interaction with Cdh1. As shown in FIG. 10A, Cdh1 and Plk1 could interact with the C terminal region of Parkin containing the RING1-IBR or IBR-RING2 domain. The RING2 domain alone, but not the RING1 domain, could interact with Cdh1. These results suggest that the IBR and RING2 domain could interact with Cdh1.

Previous studies suggest that Parkin activity is regulated by PINK1-mediated phosphorylation during mitophagy (Iguchi et al., J. Biol. Chem., 288:22019-22032 (2013)); Kane et al., J. Cell. Biol., 205:143-153 (2014); and Kondapalli et al., Open Biol., 2:120080 (2012)). The following was performed to determine if Parkin phosphorylation by Plk1 is also important for its function in mitosis. Mutation of S378 (S378A) abolished Parkin's effect toward Aurora A, Aurora B and Cyclin B1 (FIG. 9H and data not shown). Mutating other phosphorylation sites mediated by Casein kinase-1, protein kinase A, and protein kinase C did not affect Parkin's function (Yamamoto et al., J. Biol. Chem., 280:3390-3399 (2005)). Furthermore, Parkin-mediated polyubiquitination of its mitotic substrates was abolished by the S378A mutation, while it had no effect on CCCP-induced Tom20 ubiquitination (FIGS. 9I and 10B). Conversely, the S378D mutation, which mimics S378 phosphorylation, dramatically enhanced Parkin E3 ligase activity. On the other hand, mutating PINK1 phosphorylation site of Parkin (S65A) (Iguchi et al., J. Biol. Chem., 288:22019-22032 (2013)); Kane et al., J. Cell. Biol., 205:143-153 (2014); and Kondapalli et al., Open Biol., 2:120080 (2012)), although abolished CCCP-induced Tom20 ubiquitination (Geisler et al., J. Cell. Sci., 127:3280-3293 (2014)), retained basal E3 ligase activity toward its mitotic substrates comparable to WT Parkin (FIGS. 9I and 10B). The S65D mutant slightly increased Parkin E3 ligase activity toward mitotic substrates. However, it was not comparable to the dramatic increase caused by the S378D mutation. These results suggest that Plk1-mediatd phosphorylation of Parkin at S378 is another mode of Parkin activation and is important for its function in mitosis.

To further explore how Plk1-mediated phosphorylation affects Parkin function, cells were treated with BI 2536. Plk1 inhibition resulted in decreased binding of Parkin to Cdc20 (FIG. 9J). Furthermore, mutation of Ser 378 (S378A) abolished its interaction with Cdc20 during mitosis (FIG. 9K). Therefore, S378 phosphorylation is involved in Parkin's interaction with Cdc20.

### Parkin Misregulation is a Driving Event in Tumorigenesis

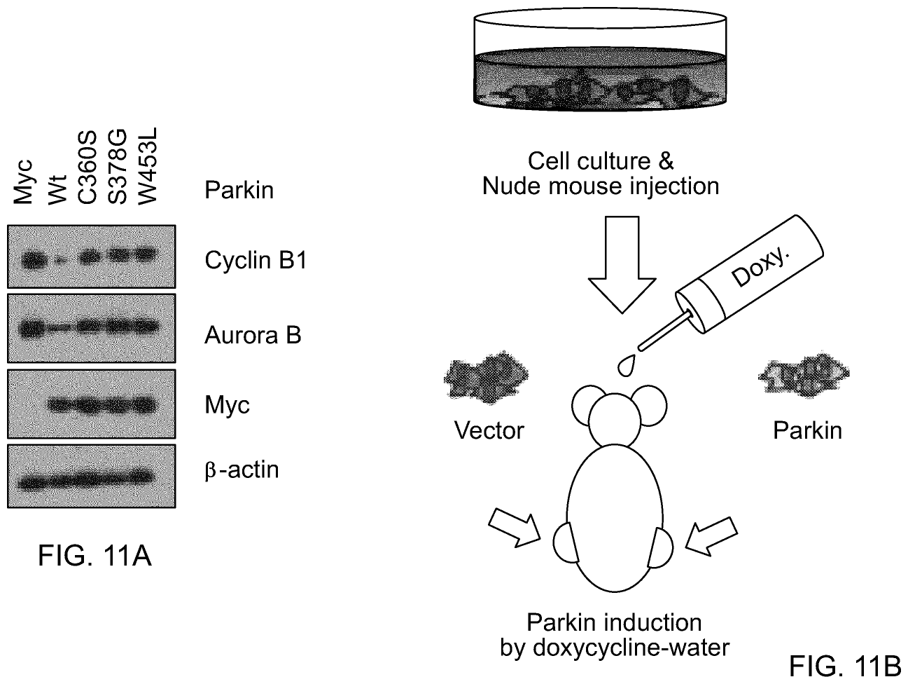

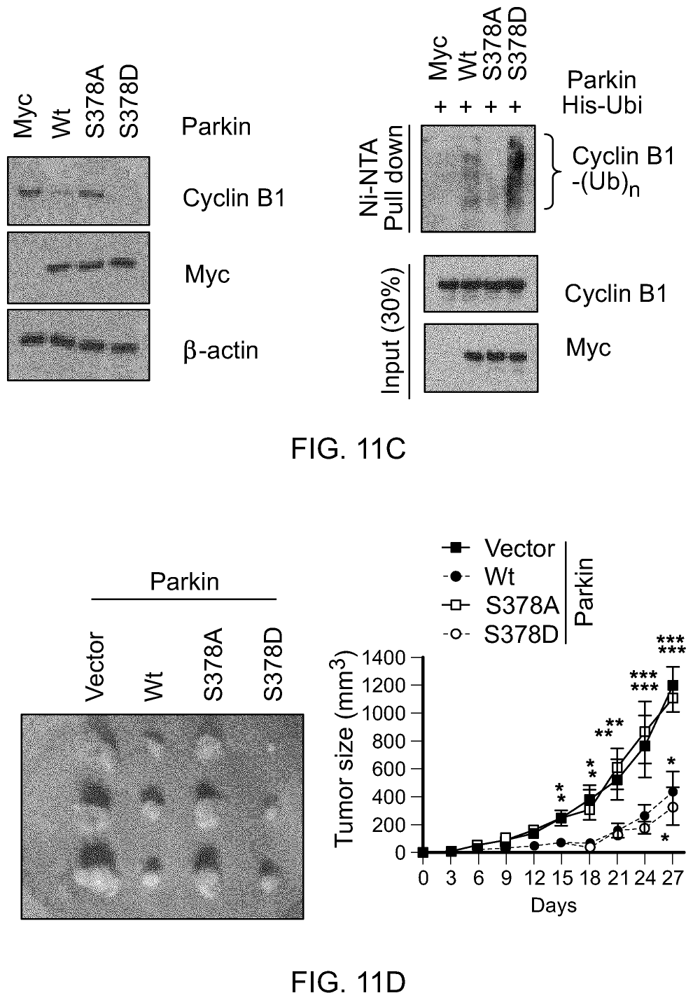

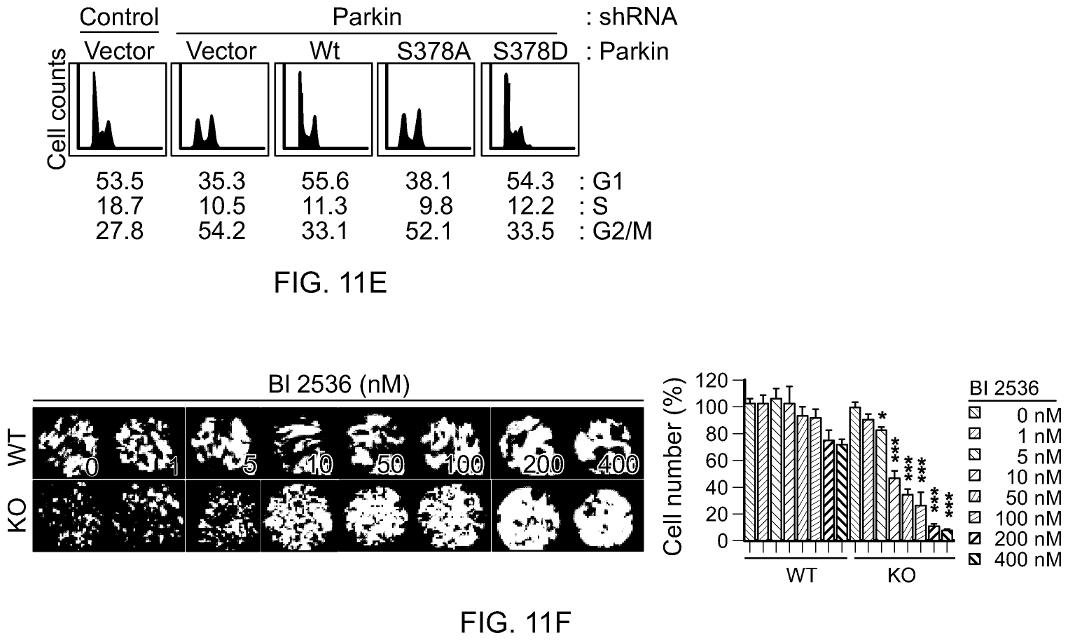

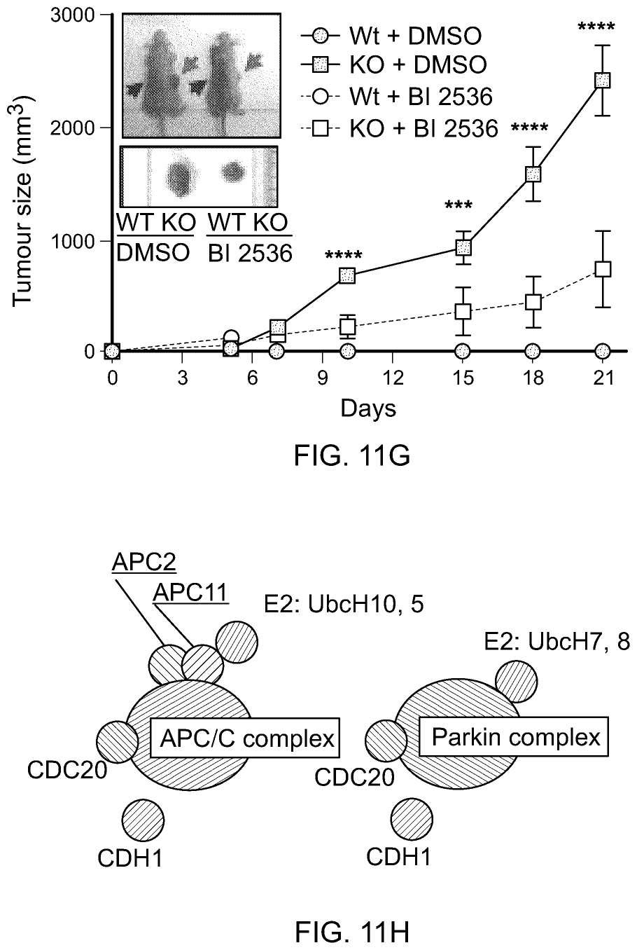

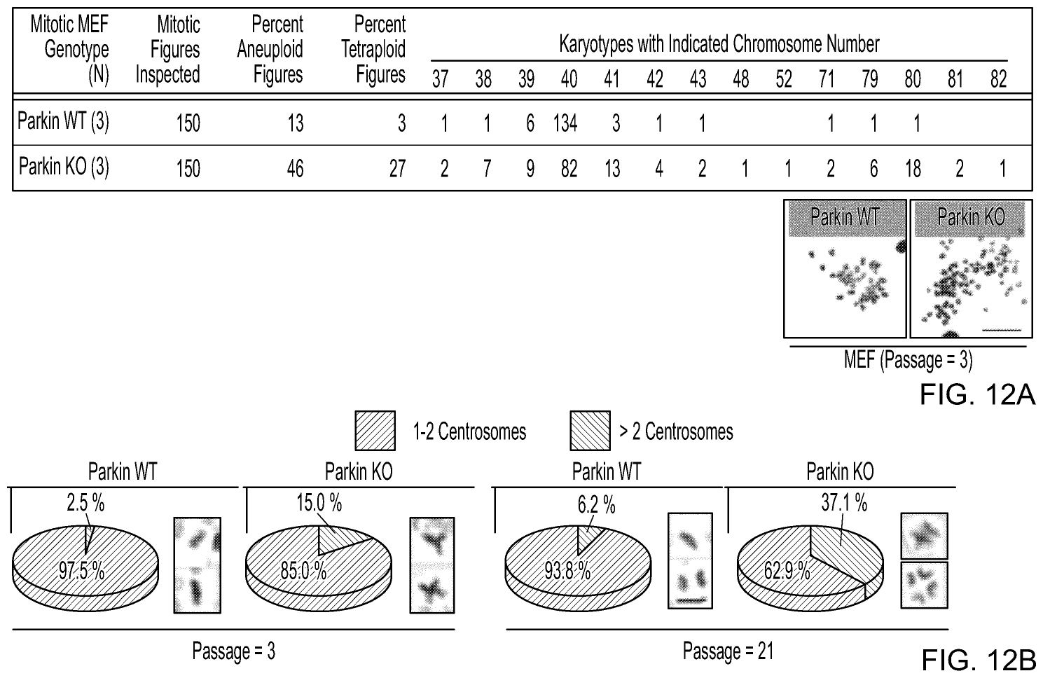

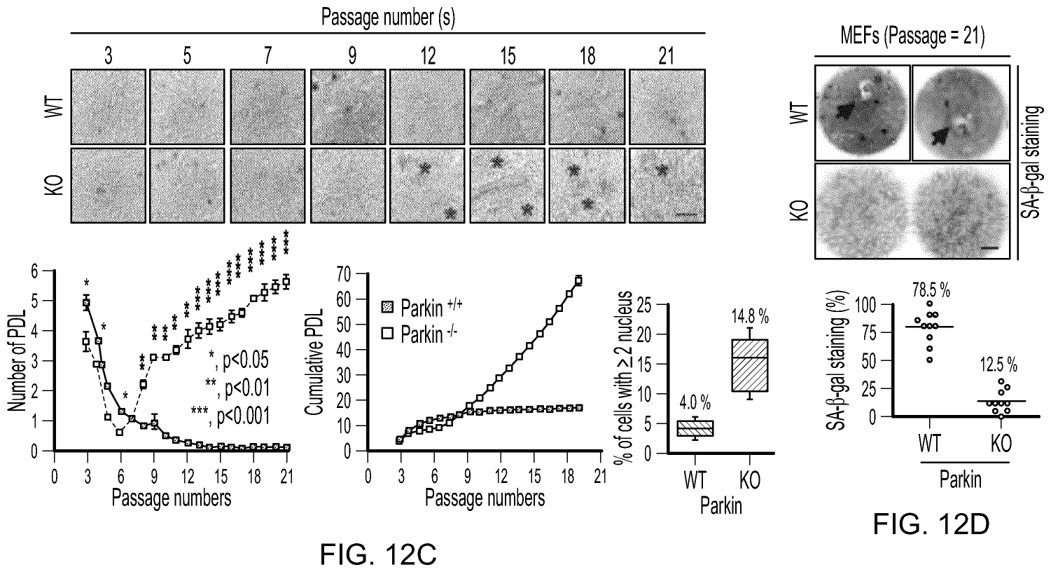

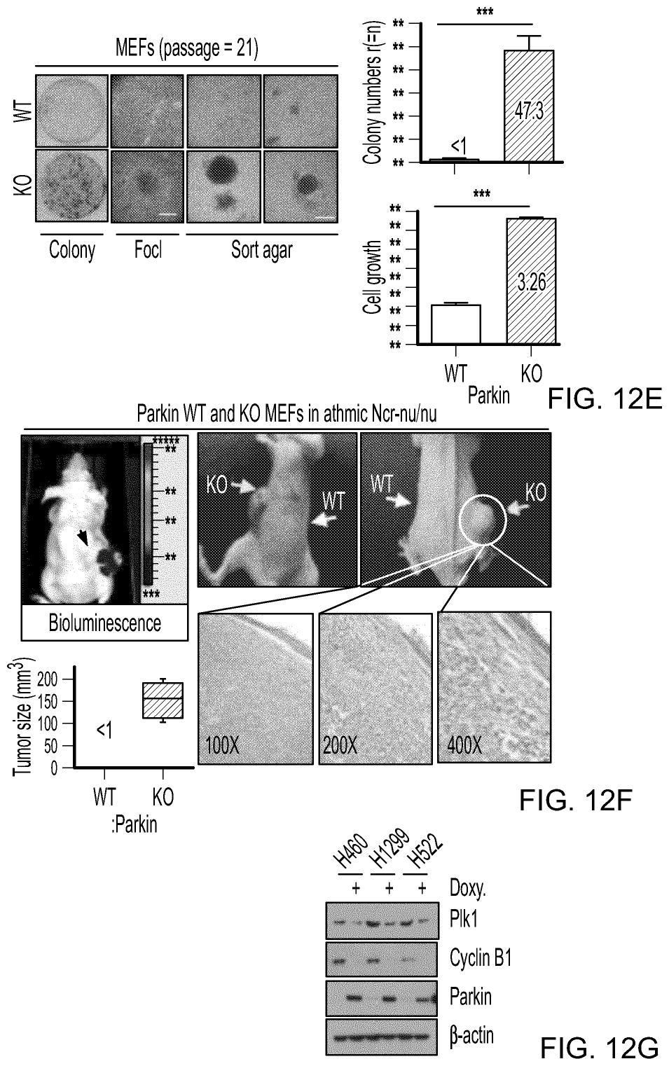

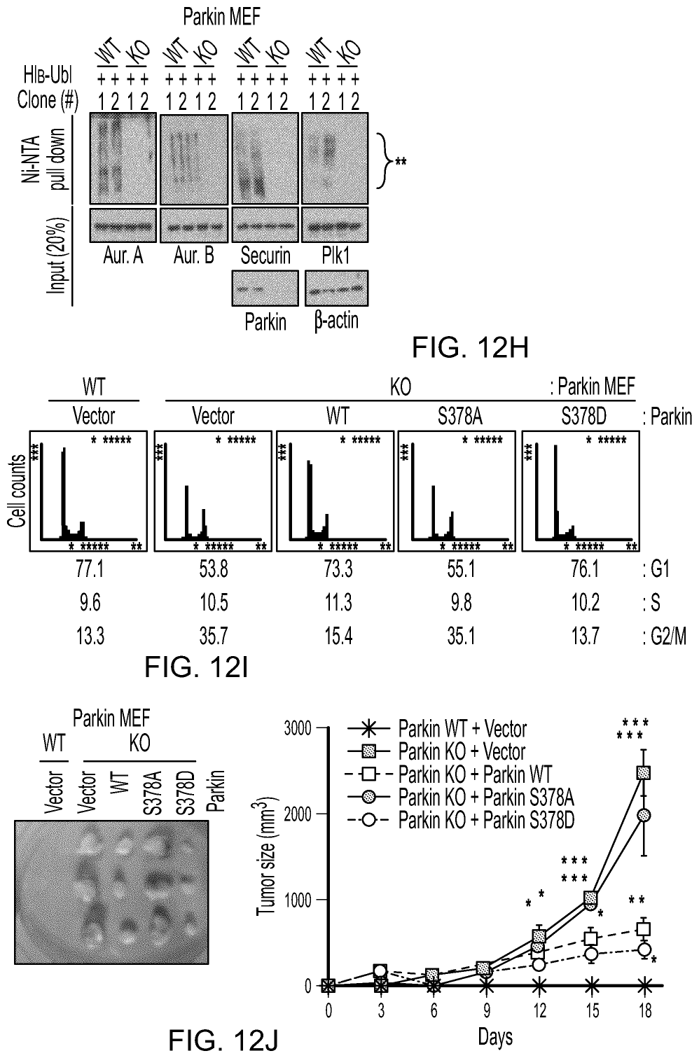

Cdh1 or Cdc20 substrates such as Plk1, Aurora A, Aurora B, Cyclin B1, and Securin are highly expressed in many types of tumors (Kim et al., Cancer Cell, 20:487-499 (2011); and Penas et al., Front Oncol., 1:60 (2011)). However, very few mutations were found in APC/C subunits (Penas et al., Front Oncol., 1:60 (2011)). On the other hand, Parkin was found to be mutated in several human cancers. Since Parkin was identified as a candidate tumor suppressor and the results provided herein demonstrate Parkin's role in regulating mitosis, it was hypothesized that Parkin has tumor suppressor function as a mitotic regulator. To further test this hypothesis, the expression of Parkin substrates in cells expressing WT Parkin or cancer-derived Parkin mutants was examined (FIG. 11A). Three tumor-associated Parkin mutations (C360S, S378G and W453L) in cBioPortal (“cbioportal.org/”) for Cancer Genomics were selected. C360 was located at the IBR Zinc region of Parkin, which was a region to interact with Cdh1. Interestingly, 5378, which was identified as a phosphorylation site by Plk1, was also mutated in cancers. The W453L mutation was found in both Parkinson's disease and cancer. These cancer-derived mutations abolished Parkin E3 ligase activity and blocked the degradation of mitotic regulators, such as Cyclin B1 and Aurora B. Parkin expression level was determined by immunohistochemical staining in 400 human lung specimens (normal and cancer) spotted on a tissue microarray (TMA; FIGS. 10C-10E). Parkin expression was lower in NSCLC samples compared to lung normal next to its cancer, but not Plk1 (FIG. 10C). Furthermore, a negative correlation was identified between Parkin and Plk1 expression (FIGS. 10C and 10F). Parkin KO MEFs exhibited more aneuploidy and polyploidy (FIGS. 12A and 12B). Furthermore, WT MEFs became senescent when cultured in vitro, while Parkin KO MEFs readily escaped senescence and became transformed (FIGS. 12C-12E). Parkin KO MEFs also became tumorigenic in vivo (FIG. 12F). These results suggest that Parkin mis-regulation is a driving event in tumorigenesis.

### Parkin is a Mitotic Regulator Functioning as a Tumor Suppressor

The following was performed to determine whether the loss of Parkin contributes to the development of human tumors. As shown in FIG. 10F, the expression of mitotic factors regulated by Parkin was much higher in all seven types of human lung cancer cell lines, while Parkin expression was low or lost in these lines in comparison to three lung normal cell lines. Doxycycline-inducible expression vectors were prepared to express Parkin and Parkin mutant forms (S378A or S378D) in Parkin-low cells to study the role of Parkin in tumorigenesis (FIGS. 11B-11E). Induction of Parkin expression in A549 cells (FIG. 11C) and other three lung cancer cells (FIG. 12G) with doxycycline resulted in decreased Cyclin B1 levels without affecting Cyclin E levels (FIGS. 11C and 12G and data not shown). In addition, Cyclin B1 became polyubiquitinated upon Parkin induction (FIG. 11C). Induction of Parkin expression with doxycycline inhibited tumor growth (FIG. 11D).

Interestingly, the S378D mutant, but not the S378A mutant, exhibited tumor suppressive function (FIG. 11D). Furthermore, Parkin-depleted cells showed G2/M accumulation, indicating a mitotic defect (FIG. 11E). These effects were rescued by reconstitution of WT Parkin and S378D mutant form but not S378A mutants. Similar results were obtained using Parkin KO MEFs (FIGS. 12C and 12H-12J). These results suggest that tumorigenicity was suppressed by Parkin expression.

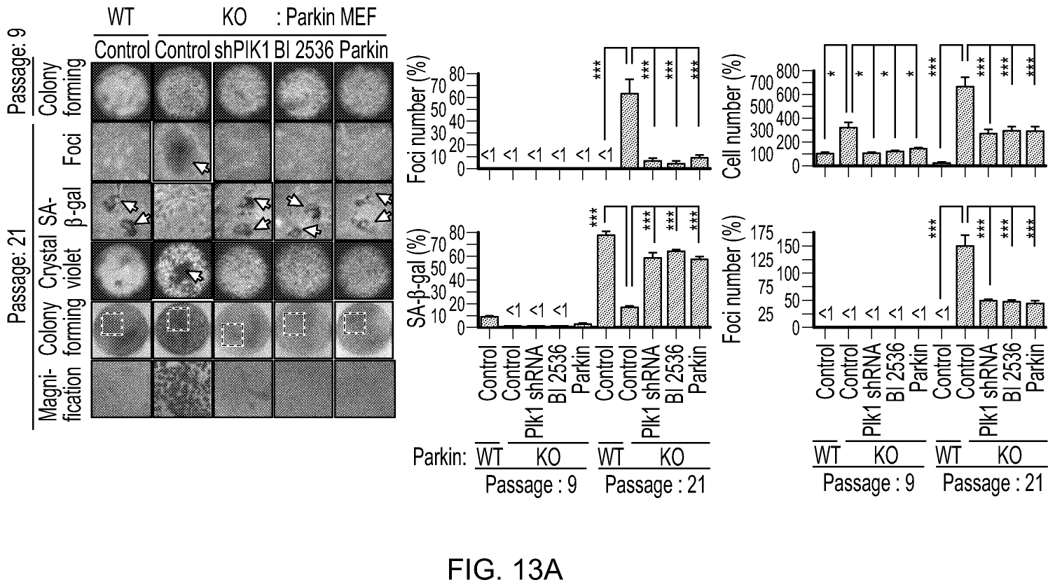

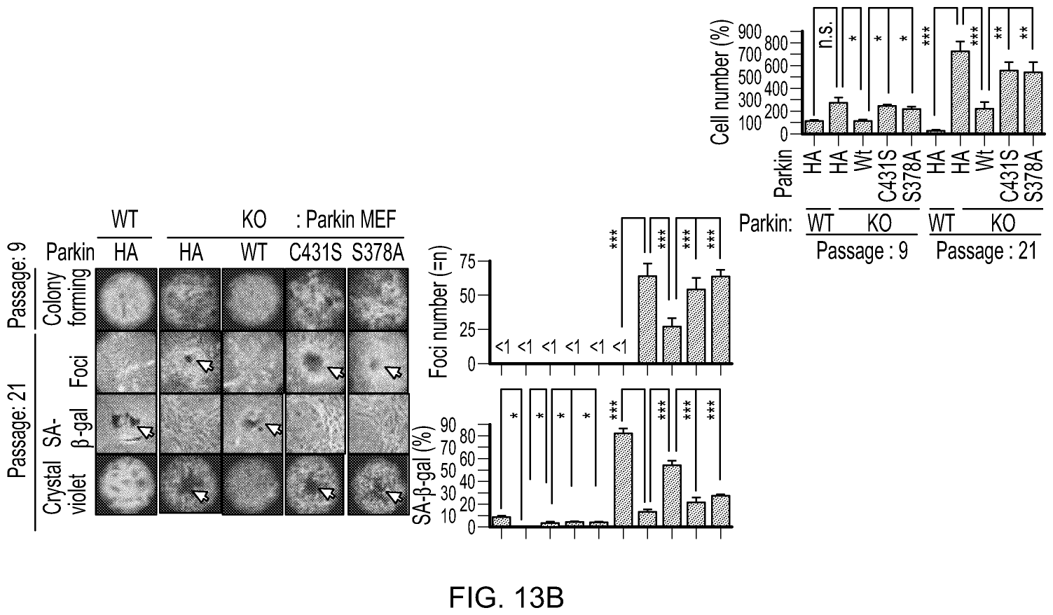

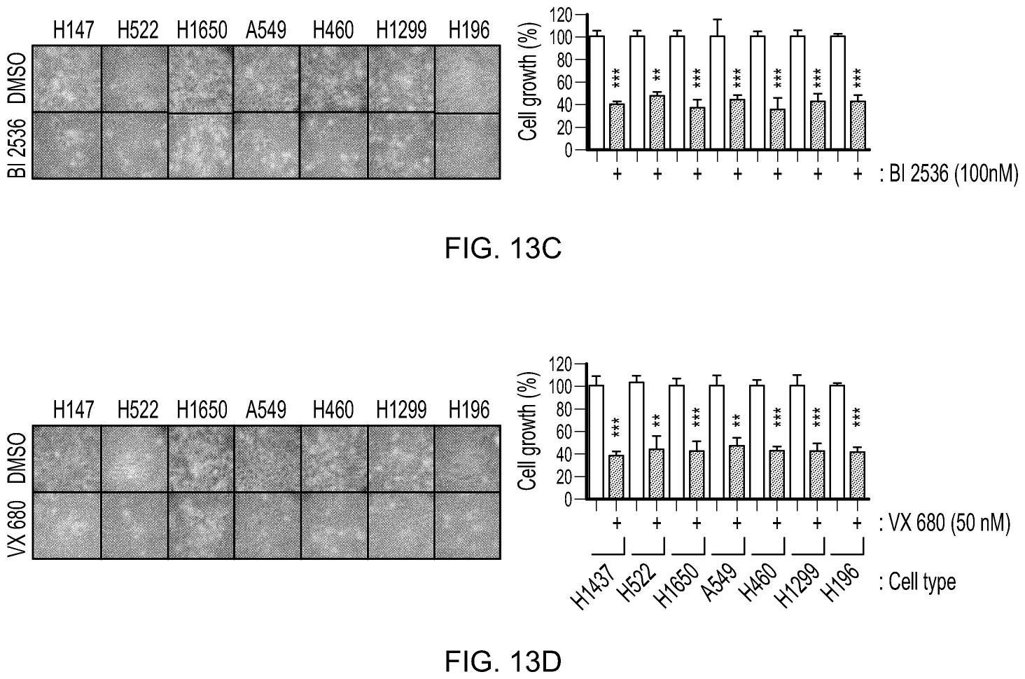

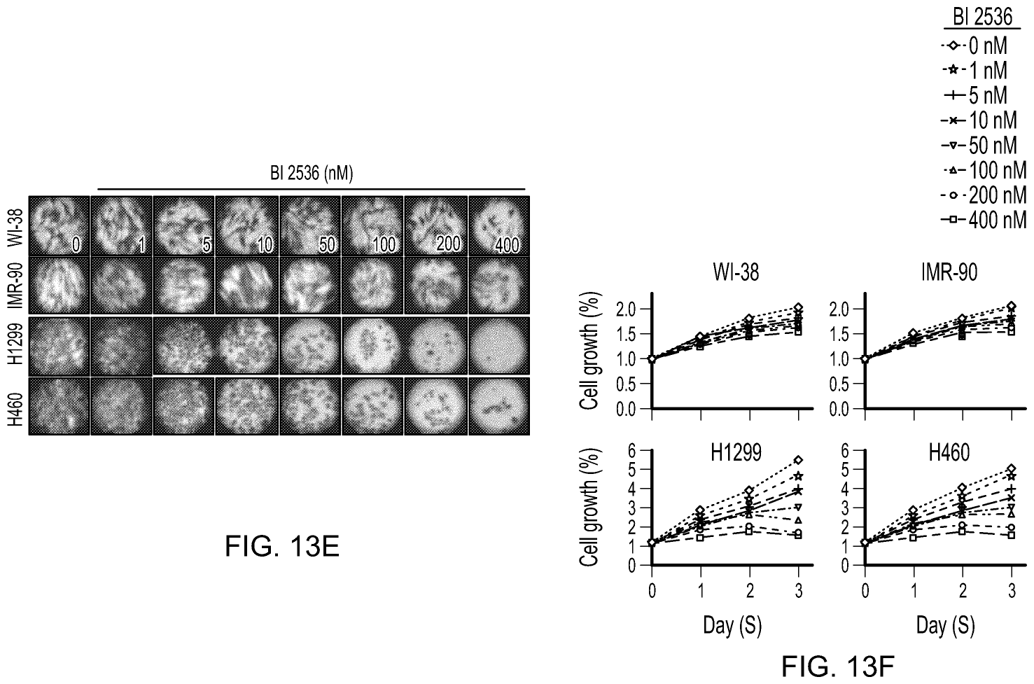

The mis-regulation of mitotic regulators in Parkin-deficient cells might provide a valuable therapeutic target. As Plk1 is overexpressed in Parkin-deficient cells, Plk1 inhibitor, BI 2536, was tested. Parkin KO MEFs were more sensitive to BI 2536 than WT MEFs, and BI 2536 inhibited transformation of Parkin KO MEFs (FIG. 11F). Furthermore, Parkin depletion induced escaped senescence, and transformation was abolished by knockdown of Plk1 or BI 2536 treatment (FIG. 13A). Transformed and down-regulation of senescence events in Parkin KO MEFs were reversed by expressing WT Parkin but not in mutants C431S or S378A (FIG. 13B). Similar results were obtained using Aurora A inhibitor, VX 680, or another Plk1 inhibitor, ON01910 in seven types of lung cancer cell lines (FIGS. 13C and 13D; data not shown). All of Parkin-deficient lung cancer cell lines but not lung normal fibroblast (WI-38 and IMR 90 cells) were significantly sensitive to Plk1 or Aurora A inhibition by BI 2536 or VX 680 (FIGS. 13E and 13F; data not shown). In addition, excellent tumor inhibition was observed with BI 2536 in vivo for tumors with Parkin-deficiency using xenograft models (FIG. 12G).

These results demonstrate that the ordered progression through mitosis is governed by two distinct E3 ligases, APC/C and Parkin, targeting mostly a common set of substrates for destruction through the shared use of Cdc20 and Cdh1 (FIG. 12H). These results also indicate that Parkin-deficiency results in overexpression of key mitotic regulators, aneuploidy, escaping from senescence, and cell transformation. Moreover, these results demonstrate that tumors with Parkin-deficiency can be treated effectively with mitotic kinase inhibitors.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating lung cancer in a mammal, wherein said method comprises:
(a) identifying said mammal as having lung cancer cells that express a reduced level of Parkin, and
(b) administering N-[2-methoxy-5-[[[2-(2,4,6-trimethoxyphenyl)ethenyl]sulfonyl]methyl]phenyl]-glycine, sodium salt (1:1) to said mammal under conditions wherein the number of lung cancer cells within said mammal is reduced.

2. The method of claim 1, wherein said mammal is a human.
